(19) United States
(12) Patent Application Publication
(10) Pub. No.: US 2011/0275891 A1
(43) Pub. Date: Nov. 10, 2011

(54) METHOD AND SYSTEM FOR IMPLANT DELIVERY

(75) Inventor: Amotz Shemi, Herzliya (IL)

(73) Assignee: SILENSEED LTD., Jerusalem (IL)

(21) Appl. No.: 13/145,392

(22) PCT Filed: Jan. 27, 2010
(86) PCT No.: PCT/IL10/00067

§ 371 Date: Jul. 20, 2011

Related U.S. Application Data
(60) Provisional application No. 61/147,548, filed on Jan. 27, 2009.

(51) Int. Cl. A61B 1/00

(52) U.S. Cl. 600/104

(57) ABSTRACT

The present invention relates to a system and method for the delivery of a treatment element, and in particular, to such a system and method in which an implantable treatment element is implanted at a target site.

### FIELD OF THE INVENTION

The present invention relates to a method and system for the delivery of a treatment element, and in particular, to such a system and method in which a non fluid implantable treatment element is implanted at a target site.

### BACKGROUND OF THE INVENTION

A medical implant may be inserted and/or implemented by variety of methods and with many different devices. The choice of delivery method largely relates to the type of the implant and the drug, the target site, the anatomical passage to such a target site, the preferred administration period, drug resistance against degradation, encapsulation options, association of the implant with other therapeutic treatments, and more. One approach widely used is the direct approach, for example, implants and/or drugs such as anti inflammatory implant, injectable immunization vaccines, hydro-gel based implants or a contraceptive implant may be delivered directly via syringe, for example in many topical delivery methods, either to provide a systemic effect or a regional effect. Another approach commonly used is an indirect approach where a drug and/or implant are delivered via a minimally invasive device or apparatus such as a guiding catheter, or an endoscope, or a laparscopic trocar, or an introducer, offering various advantages. Such advantages include access to remote tissue, flexibility enabling steering, navigating and maneuvering through curved, small and complex torturous vessels. Indirect delivery further provides for utilizing multiple access within an anatomy in a single procedure and real time visualization during a procedure by associating with real time imaging such as endoscope video, and ultrasound, or the like. Both direct and indirect implant delivery may be performed with imaging techniques such as ultrasound probes, CT, MRI, MRCP, PET, X-ray, endoscopic visible video and ultrasound, offering real time imaging and/or offline images.

A large number of delivery devices, apparatus and systems have been invented and devised for delivering drugs and/or implants into the body in both the direct and indirect approaches for both regional and systemic administration purposes. Such technologies were first developed for the delivery of fluid compositions for example, contrast solution, could be delivered to a target site using flexible drug delivery systems inserted via catheters, for example for local effect, while vaccines for example are delivered directly with a syringe and hypodermic needle for a systemic effect.

An important drug delivery application is provided with the use of an implant that may comprise an agent or drug for a controlled release along a time period that is typically a prolonged period. Such implants have been provided in various forms such as wafer, liquid including high viscous liquid, solid, or gels to introduce a plurality of different drugs and/or treatment agents having variety of pharmacodynamic, pharmacokinetic parameters including drug-release curves.

Implant delivery systems have been developed and adapted for various indications, and treatments and different target sites. For example, brachytherapy utilizes a radioactive implant known as a radiation seed that is directly inserted near the prostate for the treatment of prostate cancer. Another example is a contraceptive implant having a long release period spanning several years. Such a device is inserted with a syringe in the form of a pre-filled single-use aseptic packaging and delivery system. Other types of implant/drug delivery system include fluid and/or aqueous delivery systems, for example for intraosseous injection such as that provided for bone injection.

Other applications include subcutaneous insertion using an insertion tool such as a syringe like object. Further implant delivery systems, include a wafer implant loaded with Carmustine for intracranial implantation used following cranial surgery for debulking tumors wherein the implant is placed in the target site with the aid of tweezers.

However practitioners are still faced with many problems in delivery implant to a target site. The primary challenges faced by practitioner while delivering a medicament and/or implant include the safe delivery of the implant to the target site with minimal trauma to the target area and to tissue and/or organs along the delivery path, and the accurate precise localization of the implant at the target site. Other complicated challenges include designing the implant delivery device in accordance with the characteristics of the specific type of implant and the therapeutic agent that are used. At the same time the implant delivery system should be designed to be optimized to the passage toward the delivery site, so as to not compromise or damage the implant during the final delivery or along the passage through intermediating tissue. Other challenges include optimizing the delivery system as patient friendly and minimally invasive system, combining implantation with existing auxiliary devices and medical methods such as endoscopy, and with additional treatments including fluid injection, and utilizing the delivery with available imaging devices.

### SUMMARY OF THE INVENTION

None of the existing implant delivery systems and methods meet the challenges, requirements and needs faced by practitioners for implant delivery for implants comprising a nucleotide based agent. For example implants comprising agents selected to induce RNA interference (herein referred to as “RNAi”) mechanism.

Over the last decade RNAi was proved as a robust mode of action for gene silencing, as an approach to treat a variety of anomalies for example cancer. Recent advance in research and clinical trials of plurality of small interference RNA (herein referred to as “siRNA”) drugs raised the odds to transfer siRNA to true therapeutic treatment. siRNA is a general term for a double strand RNA molecule of 19 to 21 or about 19 to 30 nucleotides on each strand. For example oncological use of siRNA was proven experimentally in suppressing tumor growth, by targeting oncogenes or tumor growth factors. RNAi through synthetic siRNA or from expression vectors for short hairpin RNA (herein referred to as “shRNA”) are able to silence targets such as VEGF; an important factor in angiogenesis both for regenerative purposes and for pathological cases such as a tumor and diabetic retinopathy. Various delivery methods have been developed for the delivery of nucleotide-based agents including RNAi-based drugs and specifically siRNA drugs [Knocking down barriers: advances in siRNA delivery, Kathryn A. Whitehead, Robert Langer & Daniel G. Anderson. Nature Reviews Drug Discovery 8, 129-138 (2009)] including systemic administration of naked or modified siRNA, or DNA expressing shRNA (mainly used for in-vivo tests); non-viral methods including nano-particles including lipid base particles, systemic administration of liposomes encapsulating siRNA or shRNA; applying viral vectors as delivery methods; or various physical or chemical supported delivery systems like Laser Beam Gene Transfer (herein referred to as “LBGT”). However, such current delivery methods each suffer from major barriers preventing the translation into a simple therapeutic modality. Viral vectors are immunogenic some integrate into the genome and are very costly to produce. Systemic siRNA administrations induce a significant innate response and are non or poorly targeted. Liposomal systems are immunogenic, toxic, and non or weakly targeted in most cases; and in those cases which they are targeted are very complicated to manufacture. All types of siRNA/shRNA administration methods, whether systemic or through direct injection, suffer from poor targeting, immune stimulation, enzymatic degradation, toxic reactions, inability to penetrate tissue and/or cellular barriers to delivery, inefficiency of gene silencing due to non constant rate and/or short administration period, may be very expensive, or suffer from inefficiency/major side effects upon local administration such as in the case of electroporation or ultrasound mediated vascular transduction. Moreover, many of the current solutions proposed for such disadvantages are based on chemical modifications applied on the ‘naked’ form of siRNA. Such modifications might further complicate the siRNA-based treatments.

One novel approach devised by the inventor of the instant application in corresponding and pending application namely, PCT/IB2009/052778 herein incorporated by reference as if fully set forth, utilizing an siRNA or other nucleotide based agents, loaded implant designed to be inserted locally into a target site, where in some embodiments the siRNA is in a naked, unmodified form, while in some embodiments the siRNA may comprise modified forms. For example a solid tumor, where the siRNA sequence is specifically selected to silence a specific mutated oncogene, for example K-Ras mutated, that turns normal cells into tumor cells, for example in pancreatic cancer.

The primary challenges faced by practitioner while delivering an implant are the safe delivery of the implant and the precise localization of the implant at the target site with minimal trauma to the treatment area and to the path in the body selected to deliver the implant. Moreover it is desirable to minimize the time required to perform the delivery to the target site, and to minimize the invasive nature of the delivery process.

Although the background art offers many delivery systems for the delivery of implant for example syringes and balloon-based, which sometimes are aided by trocar, introducer, ports, endoscopes, guiding catheters for various types of medicaments and/or implants, they do not offer a system and method that allow for placing an implant in a target tissue, or diseased tissue without compromising the implant as described in this invention, in particular when the implant is in a non-fluid state and is comprising nucleotide based agent.

More specifically the currently available implant delivery systems and methods are not optimized to implant within a solid tissue such as a solid tumor, for example pancreatic cancer. The delivery of a solid implant is unlike that of delivery methods provided for fluid, gel, powder-like, plasma or the like compositions in particular because of the potential for harming and/or compromising the implant itself.

The delivery of liquid forms, and/or streams of micro particles and/or nano-particles and/or blowing a powder or a suspension, may be operated via very narrow opening and bear the pressures at the delivery site and along the path required to reach the target site as they assume a fluid or fluid like state. However, it cannot be assumed that a non-fluid implant or an implant that is substantially solid for example a high viscous gel can withstand the pressures applied during implant delivery. The force applied on a solid implant is with current systems might harm the surrounding tissue for example cortex neural tissue or a macular tissue, while the counter or reactive pressure applied by the target site tissue on the implant itself might affect the utility implant, for example excessive pressure and/or friction force may adversely affect a coated layer on the implant surface. Moreover, exposure to biological fluids such as stomach acids or blood, and to fluid of high pressure including high pressure blood specifically in the aorta and arteries, may compromise the implant, for example by accelerating the penetration of enzymes and/or acids into the implant surface and thereby affect the degradation of the encapsulated drug, and thereby the drug-release curve and pharmacokinetic parameters. For example penetration of blood and/or ECM serum and/or interstitial fluids, that are rich with RNAses, into an implant loaded with siRNA might result with a significant degradation in the siRNA and consequently implant performance. Therefore the requirement for delivery systems utilized for non-fluid implants and specifically implants of high sensitivity to biological degradation and to mechanical friction is immensely different than those required for delivery systems known in the art for fluid, gels and bare-metal implants.

Further constraints on a non-fluid implant delivery system may include the implant dimension itself where its size and/or dimensional characteristics may limit or affect the characteristics of the implant delivery system required. Moreover it is important to reduce the frictional forces exerted on an implant by implant delivery system walls, and/or by the surrounding tissue during implantation. For example, to avoid mechanical damage to the implant walls when implanting in hard tissue, such as bone, solid tumor, teeth, where the implant may optionally be coated by a thin layer, for example a polymeric PEG thin layer coating the implant for example to reduce interaction of the implant with proteins.

Preferably the implant delivery system and method should minimize the reaction forces between the implant and vicinity and/or exposure to biological fluids and/or chemical agents that may affect the performance of the implant. For example, in delivery system typically used for fluid and/or liquid implants or drugs to a remote target site, for example by using endoscope or catheter, the fluid can withstand the delivery distance, about 100 cm to about 200 cm, from the proximal end of the endoscopes to the target site. Injection of fluid at such a distance is reasonable and very practical, while pushing or sliding a solid implant along such a distance is undesired. Therefore a non-fluid implant must be delivered as close as possible to the target site so as to refrain from damaging the implant during delivery it is therefore important to design the method, the practice and time of loading the implant into an implant delivery system.

Another unresolved problem in non-fluid implant delivery is to minimization a non-fluid implant's interaction with biological fluids, agents and/or chemical compounds along the delivery path or during the delivery process. For example avoiding the exposing the implant to blood for example when the implantation system is inserted through the vascular system with a guiding catheter as is commonly used in interventional cardiology.

In fluid form delivery may be enabled through very narrow devices such as syringe needle of small cross section. However, the same is not necessarily true for solid implants provided in the sub-mm and mm scale, where a direct path to the target site may be inapplicable and the desired path may be long and winding path requiring for example an endoscope. Moreover in many cases of implantation of a solid implant it is desired to enable the streaming of an additional compound that is in the form of fluid.

The background art does not offer a delivery system that provides for the delivery of at least one or more non-fluid implants and specifically implant encompassing agent sensitive to biological degradation and/or sensitive to mechanical forces applied on its surfaces in a safe manner during the course of a single minimally invasive procedure. Optionally the implant may carry a plurality of different medicaments, agents, drugs, radiation sources or the like designed for a different drug-release behavior, and any combinations thereof.

The present invention overcomes the deficiencies of the background art by providing a system and method for the local delivery of a non-fluid implant to a targeted delivery site where the delivery system is minimally invasive while not compromising the implant itself during the delivery process.

Optional embodiments of the present invention provide for a system and method for a non-fluid implant delivery to a target tissue. Optionally and preferably the non-fluid implant is loaded with nucleotide agents providing for local and prolonged release of such agents, thereby providing for novel therapeutic treatments for solid cancer tumors, degenerative diseases, and regional chronic pain, and many more like indications.

An optional embodiment of the present invention provides for an implant delivery system and method that allows the implant to be deployed, delivered, placed, dropped, and/or left within a delivery canal and/or cavity such that it is not pushed, slid, blown or injected toward the tissue. Optionally this provide for the delivery of implants provided on a millimeter scale that are approximately equal to or larger than 19 gauge (outer diameter OD=1.067 mm, inner diameter ID=0.686 mm), while injection syringe in many cases are narrower, of smaller diameter. Therefore one needs to optimize for example a syringe-like system for insertion of a solid implant, of relatively large diameter.

An optional embodiment of the present invention provides for loading the implant into the system at the operation room immediately prior the delivery procedure, for example unlike Drug Elution Stent system known in the art, thereby enabling the storage conditions of the implants to be optimized at the period from manufacturing to delivery, which may be differ from the optimized storage conditions of the delivery system itself. Moreover, optionally embodiment of the present invention provides for the implant to be separated and/or detracted from a slab of implant raw material along the operation itself, enabling effective implantation of more than a single implant at the same position, and further improve the storage and logistic of the implants prior implantation.

Within the context of this application the term imaging devices may interchangeably refer to any imaging technology, and/or device producing a digital and/or physical image and/or scan, as is known in the art for example including but not limited to Magnetic Resonance Imaging or nuclear magnetic resonance imaging, or functional MRI (herein collectively referred to as “MRI”); Magnetic Resonance CholangioPancreatography (herein referred to “MRCP”), Computed Tomography, Computed Axial Tomography, CAT scan, spiral CT scan, (herein collectively referred to as “CT”); Positron Emission Eomography, PET scan (herein collectively referred to as “PET”); XRAY; ultrasound, infrared (herein collectively referred to as “IR”), laparoscopic staging. Optionally the system and method of the present invention may utilize a stand along imaging device or one incorporated into other devices or systems. For example, an ultrasound imaging device may be provided as stand alone device in the form of ultrasound probe or incorporated within other devices such as an ultrasound endoscope.

Within the context of this application the term nucleotide based agent refers to one or more RNAi agents that perform gene knockdown of message (mRNA) by degradation or translational arrest of the mRNA, inhibition of tRNA and rRNA functions; siRNA, shRNA, microRNA and non-coding RNA or the like, and Short RNAs activity on DNA, and Dicer-substrate siRNAs (DsiRNAs), and UsiRNAs and Self-delivering RNA (sdRNA), siNA, nucleotide based agents inhibiting the pre-mRNA maturation step of polyA tail addition, U1 adaptors, microRNA, aptamers, tripel-helix formation, DNAzymes, antisense, Morpholinos (PMO, phosphorodiamidate morpholino oligo); or ribozyme; or a chimeroplast; or a combination thereof.

Within the context of this application the term target site, delivery site refers to a site where the implant is targeted for placement and/or final delivery. The terms delivery site, treatment area, implantation site, target site, target tissue, may be interchangeably be referred to as target site where an implant is placed.

Within the context of this application the term treatment element may interchangeably refer to a solid polymeric matrix, elastomer, implant, wafer, fiber, fiber bundle, fiber mesh, bundle of particles, foil, drug, medicament, radiation source, energy, solid, capsule, suspension, gas, gel, plasma, liquid or a combination thereof that may provide beneficial and/or therapeutic treatment. Although the term treatment element may refer to any treatment element as described above most preferably it refers to a non-fluid treatment element comprising nucleic acids. Most preferably embodiments of the present invention for a system and method for implant delivery are adapted for the delivery of a non-fluid treatment element comprising nucleic acids implant as described in PCT Application No. PCT/IB2009/052778 termed a LODER (Local Drug EluteR) incorporated herein by reference. Preferably the LODER implant is typically made of a polymeric matrix that encapsulates at least one or more medicament and/or drug that may be released into the extracellular matrix (ECM) of the target site.

Optionally the treatment element essentially is a solid polymeric matrix, including biodegradable and/or bio-stable polymers and/or elastomers, encompassing an RNAi agent, with or without external layers, provided in the millimeter scale. In some embodiments the treatment element is provided for treating solid tissue for example including but not limited to solid tumors or the like diseased tissue. In some embodiments the implant preferably is a solid element of essentially fixed dimensions.

Optionally the system and method of the present invention may be used to deliver a treatment element to a human or animal where the drug is released to affect regionally the local area surrounding the target site, optionally within about 5 cm radius from the implant delivery site. Optionally the target site may for example include but is not limited to at least one or more of pancreas; breast; prostate; liver; gallbladder; spleen; kidney; lymph nodes; salivary glands; peridontal tissue; intra-vaginal; endocrine gland; brain; joint; bone; oral cavity; gastro-intestinal system (GI tract); biliary system; respiratory systems, heart, artery vasculature, vein; uterus, uterine cervix; fallopian tubes, ovaries, female reproductive tract, penis, gonads, male reproductive tract,; ureter or urethra; the basal ganglia, white and gray matter; the spine; active and chronic inflammatory joints; the dermis; sympathetic and sensoric nervous sites; intra osseous; acute and chronic infection sites; ear; Intra-cardiac; cardiovascular system, epicardiac; urinary bladder; parenchymal tissues; Intra-ocular; Brain tissue; Brain ventricles, intracranial space, a cavity, mouth, pharynx, esophagus, stomach, small intestine or a portion thereof, appendix, large intestine (colon) or a portion thereof, rectum or anus, auditory system, of the inner ear, vestibular system, nose, nasal conchae (also called turbinates), pharynx, larynx, trachea, bronchi, lungs, auditory tube, and the muscles of inspiration (the diaphragm and external intercostal muscles), skull, spinal canal, thoracic cavity, abdominal cavity, eye, skin, salivary glands, thyroid or pelvic cavity.

Within the context of this application the term direct delivery refers to delivery of at least one or more treatment elements or implant to a target site with the delivery system of the present invention and in some embodiments with the aid of auxiliary device. Direct delivery for example refers to delivery with optional straight and/or optionally non-flexible delivery devices.

Within the context of this application the term indirect delivery refers to the delivery of at least one or more treatment element or implant to a target site with the aid of an auxiliary device for example including but not limited to guiding catheter, catheter, endoscope, trocar, introducer, endoscope working channel, endoscope with ultrasound probe, introducer, sheath introducer, sleeve, stepper, port, or the like as is known in the art. Indirect delivery for example refers to delivery with optional flexible delivery devices, for example where delivery length is at least about 40 cm.

Within the context of this application the terms proximal, medial and distal refer to a relative gradual scale defining the relative location of objects with respect to a caregiver and/or user. For example, a proximal end portion according to the present invention refers to an assembly that is closer to a caregiver than is a distal end portion of the present invention. Similarly, a mediating member according to the present invention typically, but not limited to, is situated between its proximal and distal counterparts.

Although embodiments of the present invention may describe and refer to the delivery of an implant to a target site, the system and method of the present invention is not limited to implant delivery. Optionally the device, apparatus, and method of the present invention may be implemented and or adapted for use with a plurality of mixtures, medicaments, drugs, or the like treatment element in a plurality of optional states for example including but not limited to mixture, plasma, fluid, gases, suspensions, colloid or the like states, for delivery to a target site.

Optionally the system and method for the delivery of at least one or more treatment element may be facilitated with the use an auxiliary device for example guiding catheter, catheter, endoscope, trocar, introducer, sheath introducer, sleeve, endoscope with working channel, or the like delivery tools as is known in the art.

Optionally the delivery of at least one or more treatment element may be provided through at least one or more naturally occurring cavity for example acting as an access point to reach other location for example including but not limited to anus, vagina, urethra, oral, nasal, gastrointestinal, femoral artery, carotid artery, esophageal, auditory canal (ear), eye or the like.

Optionally delivery may be provided by penetrating the body for example by establishing an access point for example including but not limited to a port, shunt, keyhole access point, laparoscopic, artery, femoral artery or the like access point.

Optionally an auxiliary devices that may be used to facilitate the delivery of at least one or more treatment element may for example include but is not limited to Endoscopic retrograde cholangiopancreatography (ERCP), endoscopes, laparoscopes, bronchoscopes, cystoscopes, colonoscope, laryngoscopes, Sigmoidoscope, Gastroscopes, Duodenoscopes, Choledochoscope, Thoracoscope, ultrasound endoscopes, otoscope, single-use disposable scope, stereotactic medical device, catheter, sleeve, introducer or the like as is known in the art.

Optionally the system and method according to the present invention for the delivery of at least one or more treatment element may be facilitated with an imaging devices for example including but not limited to MRI, MRCP, PET, CT, IR, Ultrasound, X-ray, laproscopic staging aids, optic fibers, or the like as is known and accepted in the art.

An optional embodiment of the present invention provides for the delivery of at least one or more treatment element to a target site. Optionally, a plurality of treatment elements may be delivered to a single target site and/or to plurality of target sites that optionally are proximate. Optionally, a plurality of elements may be delivered to at least one or more target site. Optionally a plurality of treatment elements may be used having different active agents and/or pharmaceutical properties.

Optionally at least one or more treatment element may be delivered to a target site. Optionally a plurality of treatment elements may be delivered to a target site in a sequential manner and optionally spaced in a controllable manner. Optionally a plurality of treatment elements may be sequentially delivered and spaced according to a pattern. Optionally the delivery pattern of a plurality of treatment elements delivered sequential may be determined according to at least one or more parameter for example including but not limited to distance between consecutive treatment elements, target site size, location of target site, targets site shape, target site volume, type of treatment element utilized or the like.

Optionally delivery of at least one or more treatment element may be provided in a plurality of optional methods for example including but not limited to end delivery, side delivery, rotational delivery, push, pull, pull out, blow, vacuum-based, guide wire assisted, spring assisted, through an opening, through a covering, or the like.

Optionally at least one or more treatment element may be loaded into the system according to the present invention through at least one or more delivery inlet window. Optionally the system is provided with a plurality of delivery inlet windows.

Optionally at least one or more inlet loading window for loading a treatment element into the system may be provided with a means for retaining the treatment element within the delivery inlet window until deployment. Optionally retaining the treatment element may for example be provided by a number of optional means for example including but not limited to door, separator, cover, vacuum hold, biocompatible glue or the like. Optionally retaining the treatment element may for example be provided by controlling at least one or more inlet window parameters for example including but not limited to shape, size, thickness, surface treatment, materials or the like. Optionally the shape may be provided in a rectangular, conical form, or unidirectional form or the like.

Optionally at least one or more treatment element may be preloaded into the system according to the present invention. For example the delivery system according to the present invention may comprise at least one or more treatment elements in a ready to use state.

Optionally the treatment element may be provided in its delivery state according to predetermined dimensions.

Optionally the treatment element may be provided according to size associated with the target site, or target tissue that may optionally be determined during the delivery procedure, for example determine by interacting with the device or apparatus according to the present invention. For example the shape and size of the treatment element may be determined by the user during delivery using a cut out and/or cut through, procedure where the treatment element to be delivered is cut and/or deducted and/or removed from at least or more treatment element substrate for example including but not limited to a slab, block, cylinder, substrate, foil, fiber, mesh, ring and film or any combination thereof. Optionally the cut out procedure may be performed with a portion of the delivery device and/or apparatus according to the present invention.

Optionally the system according to the present invention may be provided as a disposable single time use system. Optionally the system according to the present invention may be provided from materials amenable to disposable single time use as is known and accepted in the art.

Optionally the system according to the present invention may be provided wherein at least a portion of the system is provided single time use, disposable portion. Optionally at least a portion of the system according to the present invention may be provided from materials amenable to disposable single time use as is known and accepted in the art.

Optionally the system according to the present invention may be provided in a multi-use system. Optionally the system according to the present invention may be provided in a multi-use system wherein the system may be provided from material amenable to sterilization and/or disinfection and/or enzyme-free and specifically RNAase—free techniques as is known and accepted in the art.

Optionally the system according to optional embodiment of the present invention may be further provided with a mediating assembly providing for extending the range and/or enabling flexibility and/or torque-ability between the proximal part and the distal part of the system.

Optionally the system according to optional embodiment of the present invention may be further provided with a mediating assembly providing for angled and/or radial delivery of at least one or more treatment element. Preferably mediating member is disposed between proximal and distal portions of the system according to the present invention. Optionally mediating assembly provides a controllable angle between the distal and proximal portions. Optionally the controllable angel may be selected from about 0 degrees to about 120 degrees between proximal portions and distal portion. Optionally the mediating portion may be realized according to various technologies for example including but not limited to manual, mechanical, segmentation, pneumatic, air pressure, motorized, electrical or the like technology as is known in the art. For example manual control may be provided with pulling cable. For example, air pressure and/or hydraulic control may be facilitated with a medical balloon having a present inflatable angle.

Optionally members of the system according to the present invention optionally and preferably the distal end of the system may be sealed and/or coated. Optionally coating may be provided with a biopolymer for example including but not limited to PLGA, PLA, PCL or the like as is known in the art. Optionally and most preferably coating is provided to protect an enclosed treatment element from coming into contact with the surrounding biological tissue, fluids or gasses so as to not physically, chemically or biologically interact or have an affect on the treatment element itself prior to delivery. Optionally biocompatible sealant may be applied by dipping and/or spraying at different stages for example during manufacturing and/or closer to operation.

Optionally the system according to the present invention may further comprises coating, where the coating is selected from a group including but not limited to friction reducing coating, including hydrophilic coating, cell growth enhancing, anti-microbial, anti-thrombogenic, anti-cell adhesion/proliferation, radio-opaque and the like

Optionally the system according to the present invention may further comprises and/or otherwise integrated with visual markers and/or radio-opaque materials and/or compounds for example including but not limited to heavy metals, gold, platinum, titanium, polymer enrichments, Barium Sulfate, (BaSO4), ultrasound markers, visual markers, metallic markers, IR markers, fluid enrichment elements, air enrichment elements, MRI markers, fluorine-19, IR markers, metallic markers, active markers, or the like as is known and accepted in the art.

An optional embodiment of the present invention provides for the delivery of at least one or more treatment element to a target site wherein at least one of the treatment element is provided in the form of a non-fluid treatment element comprising a nucleotide based agent, the system comprising an assembly having a distal end and a proximal end; and at least one or more opening for delivering the at least one treatment element to the target site.

Optionally the system according to the present invention may further comprises a first assembly member having a proximal end and a distal end; and a second assembly member having a proximal end and a distal end; and wherein at least one of the first assembly member or the second assembly member comprises at least one or more opening for the delivery the at least one or more treatment element.

Optionally the system according to the present invention may be provided with the treatment element comprising at least one dimension larger than 0.1 mm

Optionally the system according to the present invention further comprises a treatment element comprises at least one agent selected to induce RNA interference (RNAi).

Optionally the system according to the present invention is provided with a distal end that is provided in a shape for example including but not limited to sharp, blunt, tapered, beveled, oval, spherical, blunt with curved edges, conical, pyramidal and pyramid like having a plurality of faces, any combination thereof or the like.

An optional embodiment according to the present invention wherein the first assembly member and the second assembly member comprise a distal end having an end shape for example including but not limited to blunt, tapered, beveled, sharp, oval, spherical, blunt with curved edges, conical, pyramidal and pyramid like having a plurality of faces.

Optionally a system according to an optional embodiment of the present invention is provided wherein the distal end of the first assembly member is blunt and wherein the distal end of the second assembly member is for example including but not limited to substantially beveled, tapered and conical.

An optional embodiment according to the present invention is provided wherein at least one of the first assembly member or the second assembly member comprises a distal end shape for example including but not limited to tapered, conical and substantially beveled.

Optionally the system according to the present invention is provided where the distal end of the assembly is provided in a size selected form about 10 gauge to about 31 gauge, optionally and preferably form about 16 guage to about 25 gauge, and preferably from about 18 gauge to about 22 gauge. Optionally the distal end may be provided in a size of 10 or 11 or 12 or 13 or 14 or 15 or 16 or 17 or 18 or 19 or 20 or 21 or 22 or 23 or 24 or 25 or 26 or 27 or 28 or 29 or 30 or 31 gauge.

Optionally the system according to the present invention may be provided wherein an assembly opening comprises a cover. Preferably cover may be controllably opened or closed.

Optionally the system according to the present invention may comprise at least one or more opening provides for an implant delivery manner for example including but not limited to lateral delivery, side delivery, angular delivery, end delivery, retractive delivery, rotational delivery and pull back delivery, radial delivery, injection delivery, push, placing, deploy, dropping, deploying aspiration push by air pressure, vacuum, pressure, hydraulic, pressure differential, mechanical, manual manipulation, rotational, screw movement, helical thrust affix, stick, glue, end delivery, push, pull, pull out, blow, vacuum-based, guide wire assisted, spring assisted, through an opening, through a covering, any combination thereof or the like.

Optionally the system according to the present invention provides for configuration wherein the proximal end of the assembly facilitates control of the distal end.

An optional embodiment according to the present invention provides for an implant delivery system wherein the proximal end of at least one of the first assembly member or the second assembly member is provided for controlling the distal end the respective assembly member.

An optional embodiment according to the present invention is provided wherein the proximal end of the first assembly member is adapted for receiving the second assembly member.

Optionally the system according to the present invention is the proximal end of the assembly further comprises at least one or more treatment element inlet loading window.

Optionally the system of the present invention is provided wherein an optional assembly further comprises at least one or more treatment element inlet loading window proximally to the distal end.

An optional embodiment of the present invention is provided wherein the inlet loading window is configured to provide for receiving, vacuum pulling, magnetic pulling, holding, stowing, maintaining, retaining the at least one or more treatment element within the at least one or more inlet loading window until deployment and wherein the configuration is for example including but not limited to door, separator, cover, auxiliary device, and any combination thereof.

An optional embodiment of the present invention is provided wherein the inlet loading window comprises controllable parameters for example including but not limited to shape, dimension, size, thickness, surface treatment, materials, coating and any combination thereof.

An optional embodiment of the present invention is provided wherein the inlet loading window is provided according to dimensional parameters of the at least one or more treatment element.

An optional embodiment of the present invention is provided wherein an inlet loading window is configured to receive a treatment element wherein the treatment element is not shape specific therein provided in raw form for example including but not limited to slab, block, cylinder, substrate, foil, fiber, mesh, ring and film

Optionally the system according to the present invention is provided wherein the treatment element is implanted within the target site within a 5 cm radius.

Optionally the system according to the present invention wherein the treatment element is provided having a predetermined and consistent geometric parameters for example including but not limited to size, shape, radius, height, width, angle, thickness, volume, surface area, circumference, ellipticity, curvature, oval, polygon, curvature, hole dimension, void, waviness, roundness, layer spacing, mesh spacing, any combination thereof or the like.

Optionally the system according to the present is provided with a treatment element having a predetermined volume in the range of 0.001-8000 mm³.

Optionally the system according to the present invention is provided with a treatment element comprising geometric parameters determined by the geometric parameters of the target site.

An optional embodiment according to the present invention wherein at least one or more treatment element is provided by punching, cut out, excising, separating it from the treatment element raw form, and such excising is timely associate with the procedure of implantation.

Optionally the system according to the present invention is preloaded with the at least one or more treatment element.

An optional embodiment according to the present invention wherein the first assembly member and the second assembly are nested within one another in a manner for example including but not limited to concentric and acentric.

An optional embodiment according to the present invention wherein the first assembly member and the second assembly member are further provided with a medial portion disposed between the distal end and the proximal end in a continuous manner.

Optionally the system according to the present invention provides for an assembly that is further provided with a medial portion disposed between the distal end and the proximal end in a continuous manner. Optionally the medial portion is provided in a flexible form. Optionally the medial portion is provided in a length of at least about 10 cm.

Optionally the system according to the present invention is provided with an assembly further comprising at least one or more partition, separators and/or septum.

An optional embodiment of the present invention is provided to comprise and/or associate with at least one or more auxiliary devices for example including but not limited to a needle, guide, aspiration needle, hypodermic needle, biopsy needle, thermal needle, cryo-needle, balloon, guide wire, stapler, scalpel, anchoring ring, drill, heater, stereotactic tools, camera, imaging device, electrode, ultrasonic probe, IR transceiver transmitter/transceiver, wireless transmitter/transceiver flushing device, regional anesthesia device, cleaner, suction device, graspers, scissors, hook, ablation device, screw, pad, sticker-pad, supporting ring, embolic filters, plunger, adaptor, needle adaptor, septum/partition, net, filter, mesh, metallic mesh, ring, spring, anchors, stabilization device, stabilization part, balloon or the like in any combination thereof.

Optionally operation or control of the auxiliary device are based on at least one or more technologies for example including but not limited to mechanical, manual, electrical, optical, laser, magnetic, hydraulic, radiation, pneumatic, vacuum, air pressure, acoustic, ultrasonic and motorized, wired and wireless.

Optionally the system according to the present invention may optionally be provided with a plurality of treatment elements having at least one or more different treatment elements.

Optionally the system according to the present invention is provided with an assembly further comprises a flexible and/or bendable portion providing for introducing an angular bending and or movement from about 0 degrees to about 120 degrees about the distal and proximal ends.

Optionally the system according to the present invention the system is adapted to work with at least one auxiliary device for example including but not limited to trocar, guiding catheter, catheter, endoscope, endoscope with working channels, endoscope with ultrasound probe, borescope, introducer, stepper, sheath, port and syringe or the like.

Optionally the system according to the present invention is adapted to be operated with at least one imaging and/or scanning device for example including but not limited to video, camera, PET, MRI, MRCP, CT including spiral CT scan, IR, Ultrasound and XRAY, and laparoscopic staging.

Optionally the images and/or imaging data produced from the imaging device facilitate the delivery of the at least one or more treatment elements into the target site.

Optionally the images are used at different time relative to implant delivery for example including but not limited to during delivery, prior to delivery or following delivery, and any combination thereof.

Optionally the system according to the present invention wherein any portion of the assembly may be moved relative to another portion, for example including but not limited to rotational, lateral, distal proximal, angular, or the like in any combination or direction thereof.

Optionally the system according to the present invention provides for the proximal displacement of the distal end.

Optionally the system according to the present invention the distal end may be coupled to the proximal end with corresponding coupling members for example including but not limited to recess and latch, connectors including male connector and female connector, threading, wire, hook and loop, hook chain, braid and corresponding threading, connecting tube, snaps, magnetic, glue, or any combination thereof.

Optionally the system according to the present invention the distal end comprises a lid.

An optional embodiment of the present invention provides for a distal end that may be substantially hermetically sealed with a biocompatible sealant.

Optionally the system according to the present invention the distal end is provided as a conically shaped grasper claw that may be controllably manipulated to form an open or closed configuration comprising at least two conically shaped members and wherein each of the member correspond and engage one another to form the conically shaped grasper claw.

Optionally the system of the present invention may further comprise a conduit for delivering flowing materials of the group of fluid, gel, sol-gel, foam, suspension hydrogel, micro-particles, nano-particles, powder, solution or the like.

Optionally, the fluid may for example including but is not limited to a medicament, drug, chemotherapy agent, anti inflammation agent, antiseptic agent, pain-relief agent, anesthesia agent, corticosteroids, antiangiogenic agent, contrast solution, dyes and flushing fluid.

Optionally an assembly of the system according to the present invention may further comprises markers, for example including but not limited to visible markers, visual markers, radio-opaque compounds, ultrasound markers, fluid enrichment markers and air enrichment markers.

Optionally the system provides for radio-opaque compounds are for example including but not limited to heavy metals, gold, platinum, titanium, polymer enrichments, Barium Sulfate (BaSO4), MRI markers, fluorine-19, metallic markers, IR markers, active markers, or the like in any combination thereof.

Optionally the system according to an optional embodiment of the present invention may be provided where at least one segment of at least one part of the assembly is coated by a coating for example including but not limited to friction reducing, hydrophilic, cell growth enhancing, for example to encourage endothilialization in vascular application], anti-microbial, anti-thrombogenic, anti-cell adhesion, anti-cell proliferation, radio-opaque, non-immunogenic, non-allergic, any combination thereof.

Optionally the system according to the present invention may optionally be guided over a guide wire.

Optionally the system according to the present invention wherein the treatment element is targeted to a target site for example including but not limited to pancreas; breast; prostate; liver; gallbladder; spleen; kidney; lymph nodes; salivary glands; periodontal tissue; intra-vaginal; endocrine gland; brain; joint; bone; oral cavity; gastro-intestinal system (GI tract); biliary system; respiratory systems, cardiovascular system, artery; vein; heart, any part of the vascular system; uterus, uterine cervix; fallopian tubes, ovaries, female reproductive tract, penis, gonads, male reproductive tract, ureter or urethra; the basal ganglia, white and gray matter; the spine; active and chronic inflammatory joints; the dermis; sympathetic and sensoric nervous sites; ultra osseous; acute and chronic infection sites; ear; Intra-cardiac; cardiovascular system, epicardiac; urinary bladder; parenchymal tissues; Intra-ocular; Brain tissue; Brain ventricles, intracranial space, a cavity, mouth, pharynx, esophagus, stomach, small intestine or a portion thereof, appendix, large intestine (colon) or a portion thereof, rectum or anus, auditory system, labyrinth of the inner ear, vestibular system, nose, nasal conchae (also called turbinates), pharynx, larynx, trachea, bronchi, lungs, auditory tube, and the muscles of inspiration (the diaphragm and external intercostal muscles), skull, spinal canal, thoracic cavity, abdominal cavity, and pelvic cavity.

An optional embodiment of the present invention provides for a method for the delivery of at least one or more treatment element to a target site with an option system according to an optional embodiment of the present invention wherein an access route approach is planned toward the target site, and/or advance said auxiliary device toward target site, and visualize or image and identify said target site; and or associate the system with at least one auxiliary device; and gain access to the target site or intermediate layers toward the target site, through the devised access route with an optional system according to the present invention; and form a delivery canal within said target site; and deliver at least one or more treatment element into the delivery canal; and vacate said delivery canal.

An optional embodiment of the present invention provides for a method for the delivery of at least one or more treatment element to a target site with an option system according to an optional embodiment of the present invention wherein an auxiliary device is advanced toward target site preferably to visualize and identify the target site; and plan access route approach to the target site; and associating the system with the at least one auxiliary device; and gaining access to the target site via the access route with the system; and forming a delivery canal within the target site; and delivering at least one or more treatment element into the delivery canal; and vacating the delivery canal.

Optionally the method according to an optional embodiment of the present invention may be facilitated with at least one auxiliary device for example including but not limited to an endoscope, trocar, guiding catheter, catheter, endoscope, endoscope with working channels, endoscope comprising an ultrasound probe, stepper and introducer, or the like.

Optionally the method according to an optional embodiment of the present invention may further comprise loading at least one or more treatment element within the system.

Optionally the method according to an optional embodiment of the present invention may be provided with a system that is optionally preloaded with at least one or more treatment element.

Optionally the method according to an optional embodiment of the present invention may for the delivery of at least one or more treatment element may be performed under the guidance of at least one imaging device for example including but not limited to MRI, MRCP, CT, XRAY, IR, PET.

Optionally the method according to an optional embodiment of the present invention further comprising stabilizing the system with a stabilization device.

Optionally the method according to an optional embodiment of the present invention may further comprise bending the distal end portion of the system.

Optionally the system and method according to the present invention provides for the delivery of at least one or more treatment element to any tissue, cell or organ. Optionally and preferably the system, device, apparatus and method according to the present invention provides for delivering at least one or more treatment element to a solid tumor for example including but not 1 m

Optionally the system and method according to the present invention provides for delivering at least one or more treatment element for the treatment of cancer. Optionally a plurality forms and/or infiltrated tissue location may be treated for example including but not limited to cervical, breast, brain, lung, prostate, liver, lymphoma, uterine, urethral, salivary glands, gland, cervical or the like.

Optionally the system and method according to the present invention provides for delivering at least one or more treatment element for the treatment of ailments requiring long term medicaments for example including but not limited to cancer, AIDS, alzhymers or the like.

Optionally and preferably, the device, apparatus, system and method according to the present invention may be composed of a single and/or a plurality segments and/or materials. Most preferably the system, device and apparatus are provided from biocompatible materials as is known and accepted in the art.

Optionally the system and method according to the present invention may be depicted according to the treatment element utilized. For example, the size, dimension, and type of implant delivery system utilized according to the present invention may optionally be determined according to the treatment element utilized.

Unless otherwise defined the term about refers to a standard deviation of at least 10% such that the range is plus (+) or minus (−) 10% of the respective value.

Unless otherwise defined, all technical and scientific terms used herein have the same meaning as commonly understood by one of ordinary skill in the art which this invention belongs. The materials, methods, and examples provided herein are illustrative only and not intended to be limiting. Implementation of the system, device, apparatus and method of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D are a schematic illustration of an optional system according to the present invention.

FIGS. 2A-B are schematic illustrations of an optional proximal portion of the system according to an optional embodiment of the present invention for the delivery at least one or more treatment element;

FIGS. 3A-F are schematic illustrations of an optional distal portion of the system according to an optional embodiment of the present invention provided for the delivery of at least one or more treatment element that is preloaded within the distal portion of the system according to an optional embodiment of the present invention;

FIGS. 4A-J are schematic illustrations of an optional distal end of the system according to an optional embodiment of the present invention provided for the delivery of at least one treatment element loaded through a delivery inlet loading window according to an optional embodiment of the present invention for;

FIGS. 5A-B are schematic illustrations of optional distal portions of the system according to the present invention provided for the retractive and/or pull out delivery of at least one treatment element according to an optional embodiment of the present invention in a 2 to 1 manner;

FIGS. 6A-F are schematic illustrations of optional distal portions of the system according to an optional embodiment of the present invention provided for the delivery of at least one treatment element, using a sharp end and avoiding a shaft needle;

FIGS. 7A-C are schematic illustrations of another optional distal portion of the system according to optional embodiment of the present invention using a sharp end and avoiding a shaft needle;

FIG. 8A-C are schematic illustrations of optional distal end of the system according to the present invention provided for the delivery of at least one treatment element via lateral and/or side delivery according to an optional embodiment of the present invention;

FIGS. 9A-B are schematic illustrations of an optional treatment element provided for delivery at direction angular to the system insertion direction, including radial delivery of at least one treatment element according to the present invention.

FIGS. 10A-B are schematic illustrations of an optional stabilization device for the delivery of at least one or more treatment element according to an optional embodiment of the present invention with a stabilization device;

FIGS. 11A-E are schematic illustrations of an optional device for the delivery of at least one or more treatment element according to an optional embodiment of the present invention in a direct manner;

FIGS. 12A-D are schematic illustrations of an optional treatment element delivery device according to the present invention provided for the delivery of at least one treatment element loaded in real time through a distal delivery inlet according to an optional embodiment of the present invention;

FIG. 13 is a schematic illustration of an optional distal portion of the system according to an optional embodiment of the present invention provided for the delivery of at least one treatment element with the aid of a guide wire.

FIG. 14 is a flowchart of an optional method for the delivery of at least one or more treatment element at a target site according to the present invention.

FIG. 15 is a flowchart an optional method for the delivery of at least one or more treatment element according to the present invention.

### DESCRIPTION OF THE PREFERRED EMBODIMENTS

The principles and operation of the present invention may be better understood with reference to the drawings and the accompanying description.

The following reference label lists is a legend of the numbering of the application illustrations that are used throughout the drawings to refer to objects having similar function, meaning, role, or objective.

10 Treatment Element, implant;

10h treatment element housing;

15 directional arrow;

20 First assembly member;

22 First assembly member proximal end;

24 First assembly member distal portion;

24t First assembly member distal end;

24rc Second assembly recess cover;

24p pre-implantation site;

28 shaft lock

30 Second assembly member;

32 Second assembly member proximal end;

34 Second assembly member distal portion;

34t Second assembly member distal end;

34r Second assembly member distal end recess;

34rc Second assembly member distal end recess cover;

34c Second assembly member distal end cover;

34p pre-implantation site;

36 second assembly first conduit;

38 second assembly second conduit;

40 mediating member;

50 implant delivery system;

52 syringe-like implant delivery system;

60 anchoring/stabilizing device;

62 guide wire;

64 guide wire conduit;

66 balloon inflating tube;

101 distal end portion;

101p proximal portion of distal end portion 101;

101d distal portion of distal end portion 101;

106 external housing distal portion;

108 external housing distal tip;

112 Puncturing element, needle;

116 Delivery guide shaft;

116d Delivery guide-shaft distal tip adaptor;

118 adaptor, needle adaptor;

119 adaptor displacer;

120 treatment element inlet loading window;

122 treatment element outlet window;

124 Implant delivery outlet cover;

126 loading window cover;

128 treatment element holder;

220 plurality implant loading window assembly;

130 Septum, separator;

150 proximal end portion;

151 Proximal end portion housing;

152 Proximal housing needle Plunger/controller;

154 Proximal assembly housing distal end proximal end;

156 Proximal assembly delivery guide shaft controller;

156l proximal assembly delivery guide shaft lock;

300 Patient;

302 Stomach;

304 Pancreas;

306 treatment element delivery route;

308 Target site and/or tissue;

310 Endoscope;

312 Endoscope distal end;

314 Endoscope proximal end;

318 endoscope conduit;

316 Endoscope working channel access point;

320 Imaging device;

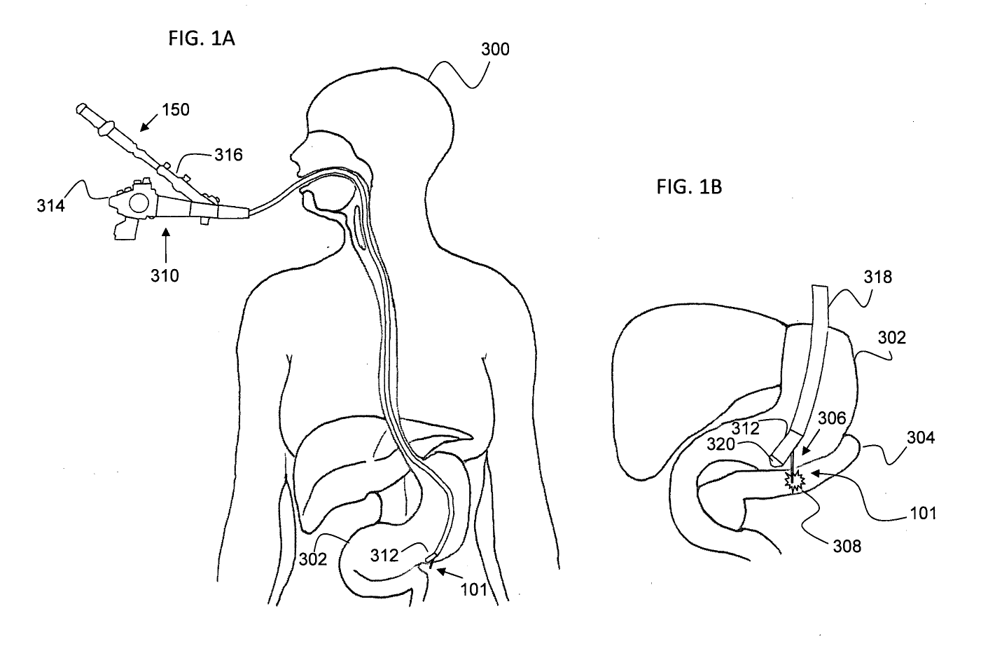

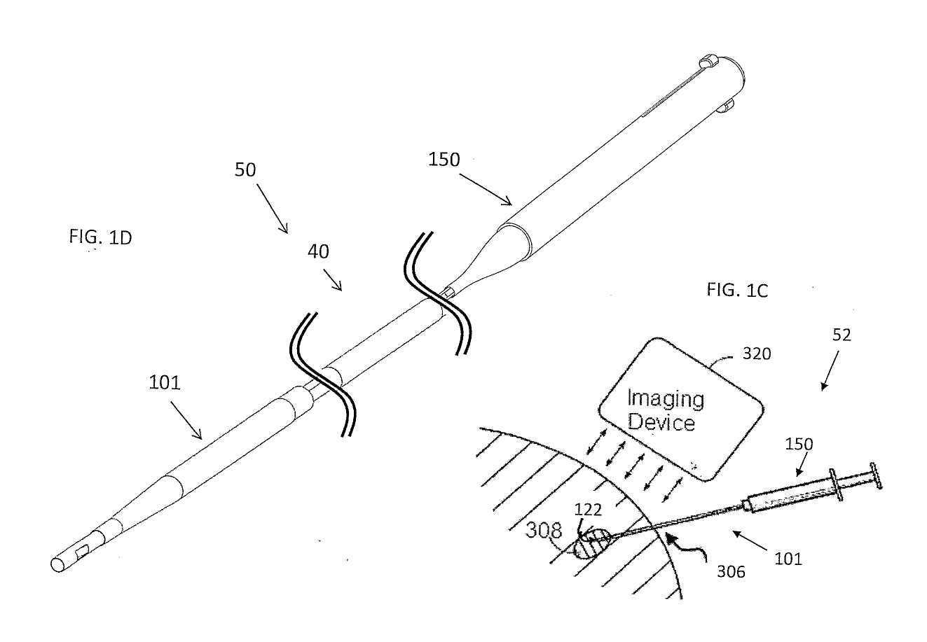

FIGS. 1A-B provide a schematic illustration of an optional implant delivery system according to the present invention wherein delivery is facilitated via channeling device including but not limited to guiding catheter and/or via a remote visualization device as is known and accepted in the art for example including but not limited to a scope or an endoscope. Although FIGS. 1A-B depict an optional embodiment of the system and method of the present invention with a gastrointestinal endoscope 310 with an ultrasound imaging probe 320 for the delivery of at least one or more treatment element 10, most preferably in the form of a LODER, to a pancreatic target site 308, the preset invention is not limited to use with an endoscope and may be utilized with a plurality of optional auxiliary devices for example including but not limited to trocar, guiding catheter, catheter, endoscope, endoscope with working channels, endoscope with ultrasound probe, introducer, stepper, port, syringe, flexible sleeve, micro-catheter (usually used for neurovascular treatments), flow directed catheters, medical hollow shaft or the like as is known and accepted in the art.

Preferably a plurality of endoscope types may for example be used with the implant delivery system according to optional embodiments of the present invention where the type of endoscope utilized is dependent on the target site 308, the passage toward such a site, and the chosen implant delivery approach, for example endoscopic or laparoscopic or the like. For example scopes that may be used for facilitating the implant delivery with the system and method according to optional embodiment of the present invention may for example include but are not limited to Endoscopic retrograde cholangiopancreatography (ERCP), endoscopes, laparoscopes, bronchoscopes, cystoscopes, colonoscope, laryngoscopes, sigmoidoscope, gastroscopes, duodenoscopes, choledochoscope, thoracoscope, ultrasound endoscopes, otoscope, single-use (disposable) endoscope, Fertiloscope or the like as is known in the art.

Optionally endoscope 310 comprises a controller 314 for controlling the movement and function of endoscope 310 within the accessed anatomy, for example, a portion of the GI tract accessed through the oral cavity and progressing to the stomach 302 via the esophagus, as shown. Optionally endoscope 310 comprises a working channel (not shown) that may be accessed through a working channel entry point 316. Optionally the implant delivery operation includes a method of NOTES (Natural Orifice Transluminal Endoscopic Surgery) as is exampled in FIG. 1A-B where in this example the stomach wall is firstly pierced, enabling the operation beyond the GI tract while for example avoiding general surgery.

FIG. 1A provides a perspective view while FIG. 1B provides a close up view of an optional target site 308 within the pancreas 304 accessed through access route 306, where for example a portion of stomach wall 302 is penetrated to gain access to the target site 308. Endoscope 310 provides a working channel (not shown) through which the implant delivery system 50 according to an optional embodiment of the present invention may be advanced, and in general utilized. Implant delivery system 50 comprises a first assembly member 20 and a second assembly member 30 each comprising a proximal and distal end that may optionally and preferably be associated with one another forming a proximal end portion 150 and a distal end portion 101. Optionally the first and second assembly members of implant delivery system 50 may further comprise a mediating member (not shown) optionally flexible provided to couple and/or optionally assisting to transfer push and pull longitudinal movements and/or rotational torque transferring movements otherwise associate the distal end and proximal end respectively therein preferably forming a continuous flexible mediation portion disposed between proximal end portion 150 and distal end portion 101.

Optionally and preferably proximal end portion 150 may be associated endoscope 300 through working channel entry point 316. Optionally and preferably distal end portion 101 may be threaded from entry point 316, through the length of working channel (not shown) to endoscope distal end 312 of endoscope 310 optionally comprising an ultrasound probe 320 wherein it functions to provide ultrasonic imaging of the targeted implantation site 308 and for planning access route 306.

Most preferably proximal end portion 150 provides for manipulating and controlling distal end portion 101 and comprises at least one or more controllers for controlling distal end portion 101. Optionally control of distal end portion 101 is provided via manual, mechanical, electrical, optical, laser, magnetic, hydraulic, pneumatic, motorized, ultrasonic, acoustic, wired and/or wireless technology, a combination thereof or the like.

Most preferably distal end portion 101 comprises at least one or more auxiliary devices provided for facilitating the delivery of at least one or more treatment element 10 (not shown), for example including but not limited to a non-fluid implant comprising RNAi-based agent within a target site 308, for example including but not limited to a pancreatic solid tumor, as shown in FIG. 1B.

FIG. 1C provides a schematic illustration of an optional implant direct delivery system 52 according to the present invention comprising a proximal end portion 150 and a distal end portion 101 that are preferably are coupled and or otherwise associated and provided in the form of a syringe-like apparatus for the delivery of at least one or more treatment element 10, most preferably in the form of a LODER, to a target site 308 through access route 306. Optionally and preferably implantation of treatment element 10 to target site 308 is facilitated with an imaging device 320, for example including but not limited to MRI, CT, X-ray, PET, ultrasound or the like imaging device as is known and accepted in the art.

FIG. 1D provides schematic illustrations of an optional system 50 for the delivery of at least one or more treatment element delivery comprising proximal end portion 150, mediating portion 40 and distal end portion 101. Optionally system 50 may be associated with an auxiliary device for example including but not limited to a catheter, endoscope, trocar, or the like, to facilitate implant delivery according to the present invention.

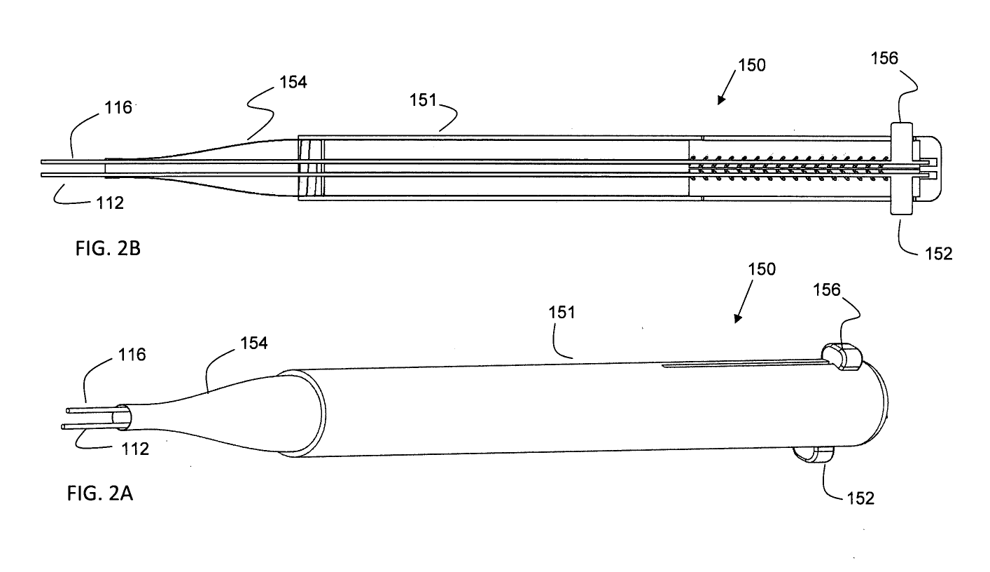

FIG. 2A-B are schematic illustrations of an optional proximal end portion 150 of an optional implant delivery system 50 according to the present invention for the delivery of at least one or more treatment elements 10 (not shown). Optionally proximal end portion 150 may be used with an auxiliary device for example including but not limited to a trocar, guiding catheter, catheter, endoscope, endoscope with working channels, endoscope with ultrasound probe, introducer, stepper, port, syringe or the like, to facilitate implant delivery according to the present invention.

FIG. 2A provides a perspective view of proximal end portion 150 comprising first assembly member proximal end housing 151 and connector 154 optionally for coupling and or otherwise associating with a distal and/or medial portion of the system 50. Optionally connector 154 may be adept for connecting with an auxiliary device such as an endoscope 310 having a working channel entry point 316. Optionally housing connector 154 may be disposed on at least one or both of the first assembly member or second assembly member according to optional embodiments of the present invention. Optionally, connector 154 may be provided in a plurality of optional controllable connector forms to facilitate coupling and/or association for example including but not limited to threading, snaps, male connector, female connector, recess and latch, lock and key, recess and cogs, unidirectional lock or the like as is known and accepted in the art.

Preferably proximal end portion 150 comprises at least one or more controllers optionally provided for controlling the distal end portion 101 (not shown) of at least one of or both first assembly member or second assembly member. For example mechanical controllers 152, 156 may be provided for controlling and/or manipulating the distal end of the second assembly member. Optionally controllers 152, 156 may provided control using optional technologies for example including but not limited to mechanical, hydraulic, pneumatic, wired and/or wireless technology, threading, spring based a combination thereof or the like.

For example, second assembly member controllers 152 and 156 are preferably associated with a corresponding member disposed in the distal end of the second assembly member for example a delivery shaft-guide 116 and needle 112 both optionally disposed within the second assembly member according to an optional embodiment of the present invention. Optionally second assembly member controller 152 may be provided as a longitudinal, spring based mechanical controller of a needle 112 disposed at the distal end of the second assembly member. Optionally second assembly member mechanical controller 156 may also be provided in the form of a longitudinal spring based mechanical controller of second assembly member delivery shaft-guide 116 disposed in the distal end of the second assembly member.

Although the preceding controllers 152 and 156 were described as both controlling the second assembly member of the present implant delivery system it is to be understood that controller 152 and/or 156 may control any portion of the distal end of the first and/or second assembly member according to the present invention. For example while controller 152 may control a member of the second assembly member at the distal end, controller 156 may control a member of the first assembly member at the distal end and/or a mediating member

FIG. 2B provides a sectional view of proximal end portion 150 showing optional second assembly member spring based mechanical controllers 152 and 156 as well as connector 154, which is optionally provided for adapting the dimension of connected parts, for example in a tapered and/or conical shape, disposed on the distal portion of the proximal end of second assembly member, as described in FIG. 2A above, optionally provide for controlling a second assembly member delivery shaft-guide 116 and needle 112 functioning along the distal end of the second assembly member.

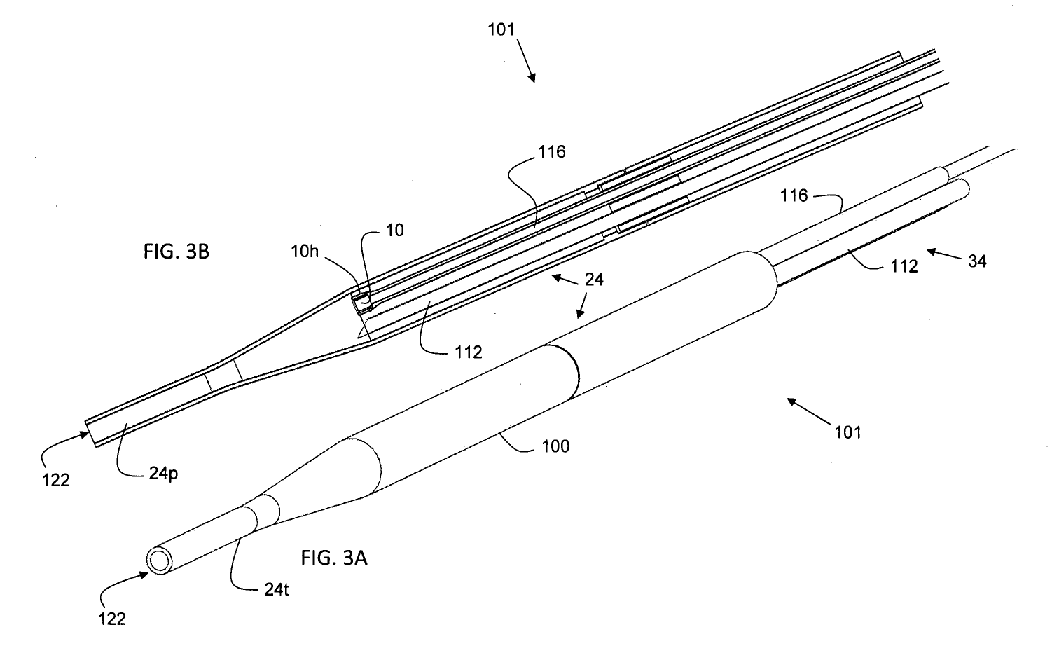

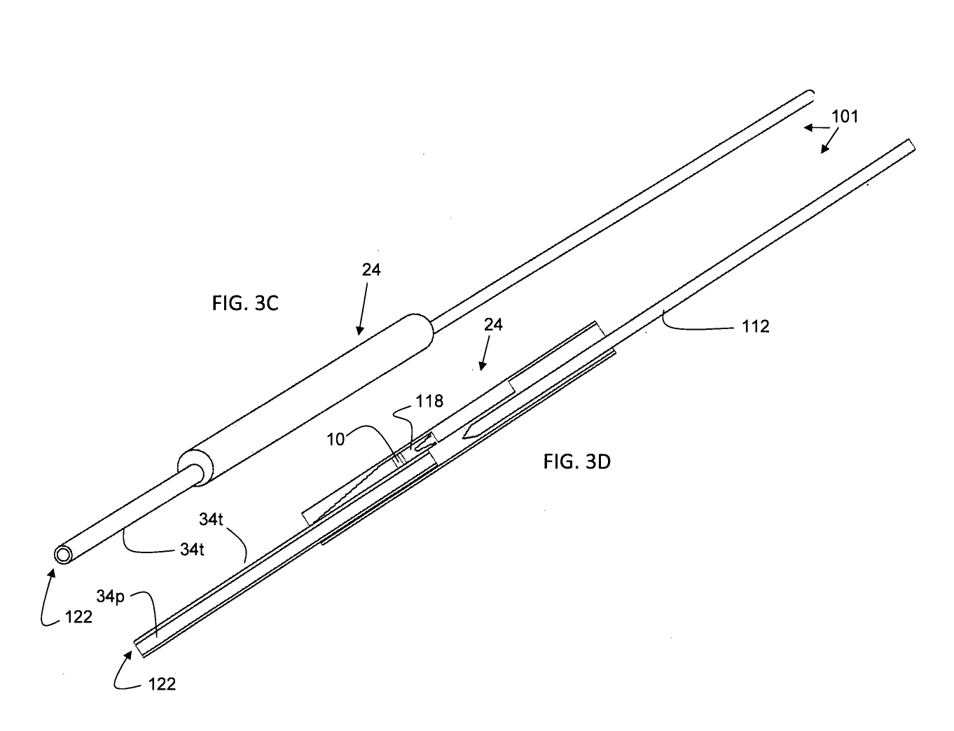

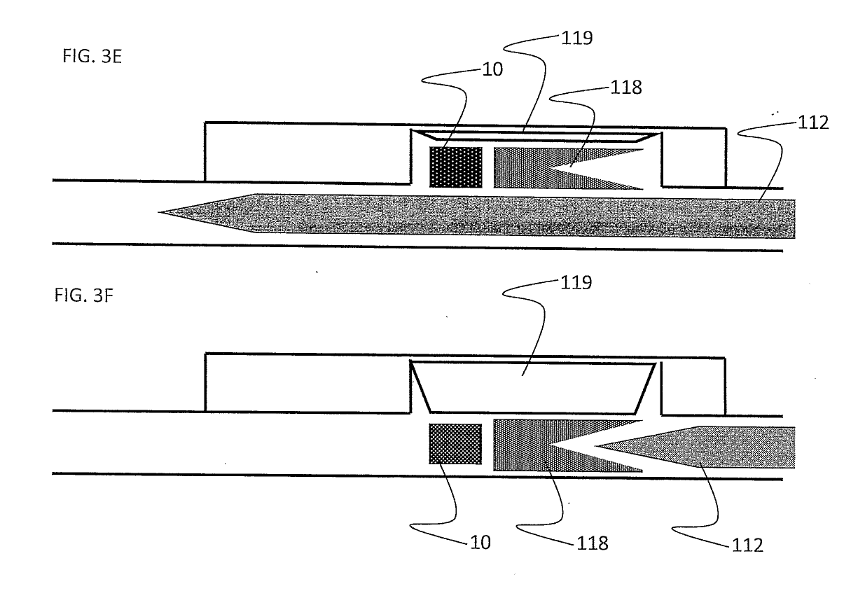

FIGS. 3-10 depict optional embodiments of distal end portion 101 comprising at least a portion of the distal ends of the first assembly member and the second assembly member according to an optional embodiment of the present invention. Optionally distal end portion 101 may be used with an auxiliary device for example including but not limited to a catheter, endoscope, trocar, or the like, to facilitate implant delivery according to the present invention.

Optionally distal end portion 101 comprises distal portion of first assembly member 24 comprising a housing 100 forming a primary lumen comprising second assembly member distal portion 34. Optionally second assembly member distal portion 34 comprises at least one or more auxiliary devices for example including but not limited to needle 112, shaft-guide 116, aspiration needle, hypodermic needle, biopsy needle, thermal needle, cry o-needle, balloon, guide wire, stapler, scalpel, anchoring ring, drill, heater, stereotactic tools, camera, imaging device, electrode, ultrasonic probe, IR transceiver transmitter/transceiver, wireless transmitter/transceiver flushing device, regional anesthesia device, cleaner, suction device, graspers, scissors, hook, screw, pad, sticker-pad, supporting ring, embolic filters, plunger, adaptor, needle adaptor, septum/partition, net, filter, mesh, metallic mesh, ring, spring, anchor or the like

Optionally and preferably second assembly member distal portion 34 comprises needle 112, shaft-guide 116 for facilitating the delivery of at least one or more treatment element 10, most preferably provided in the form of a non fluid implant comprising RNAi based agent selected specifically to affect the tissue at a target site 308.

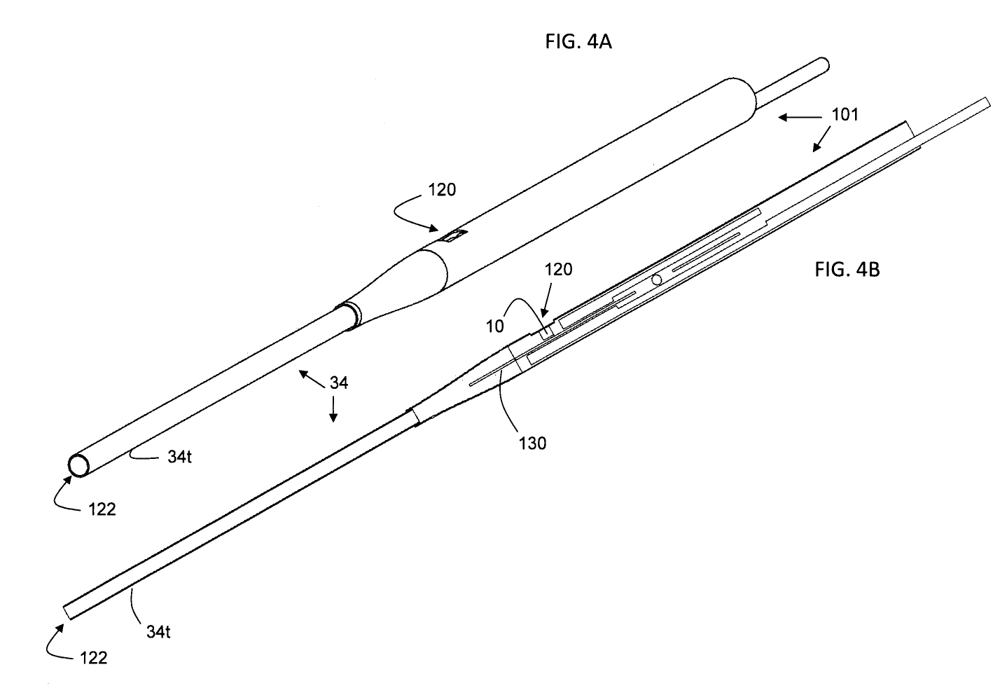

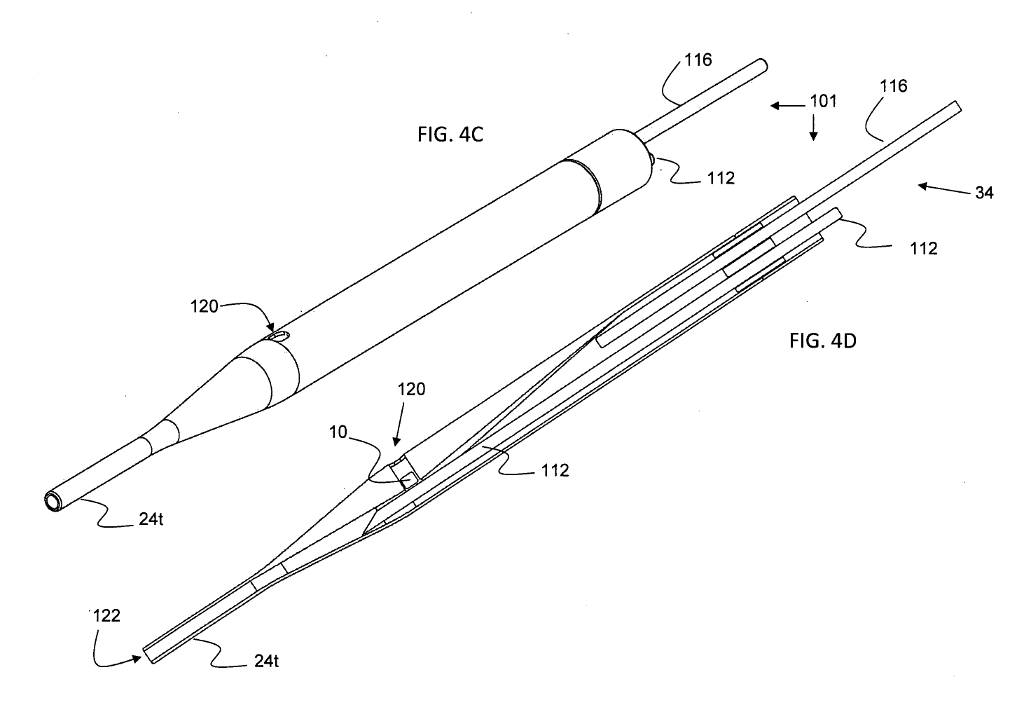

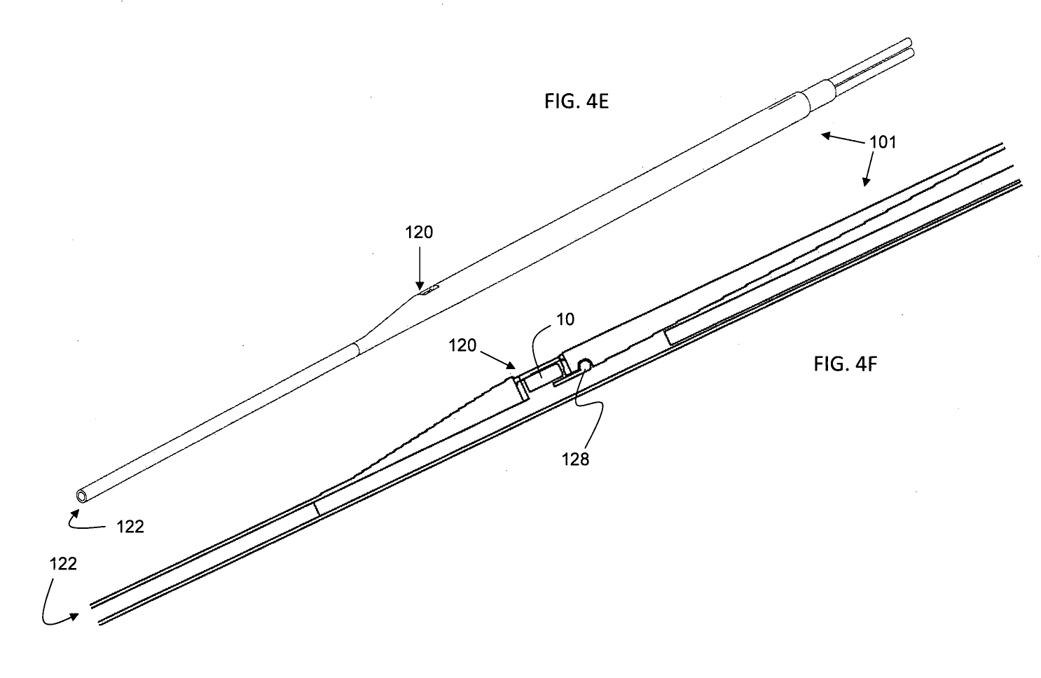

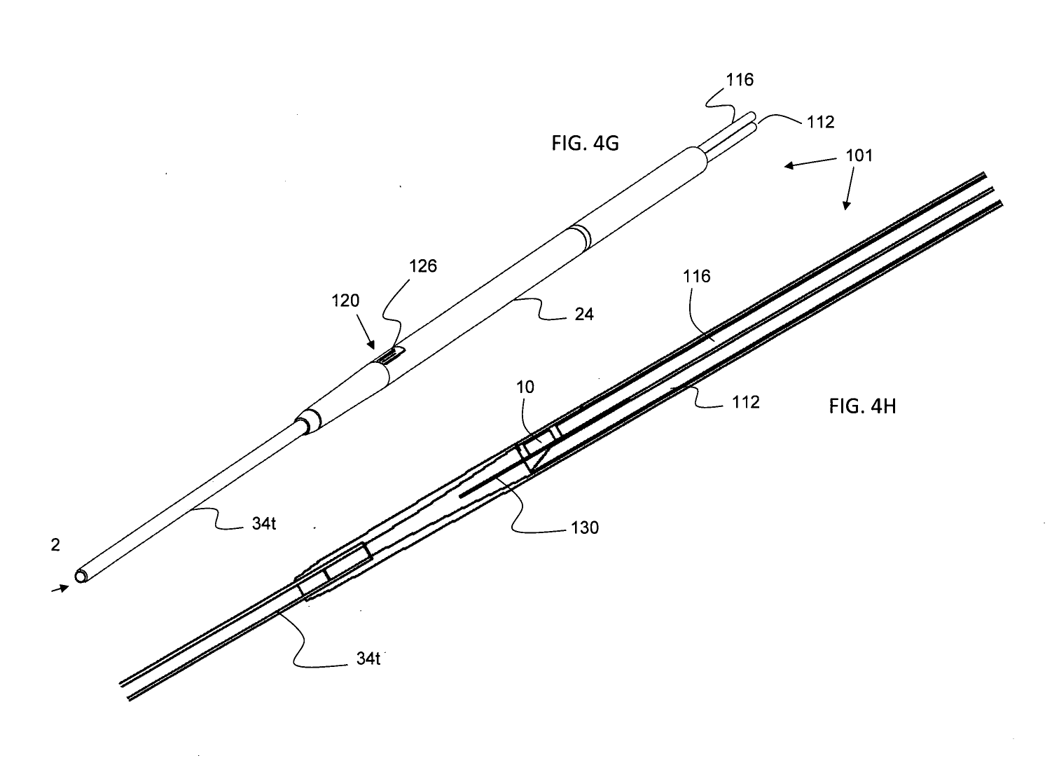

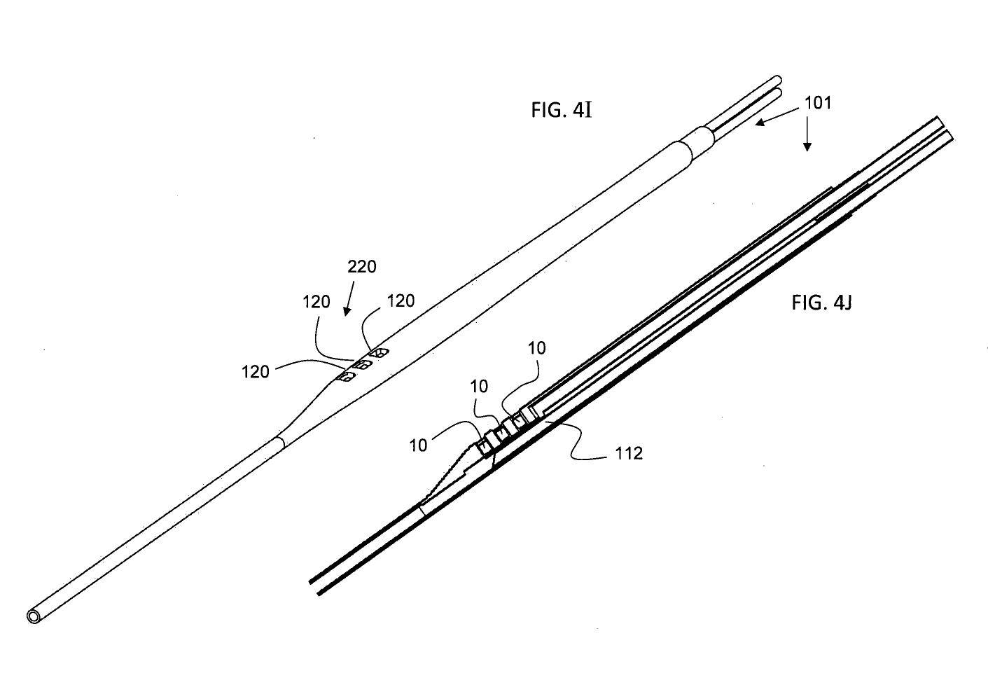

Optionally treatment element 10 may be preloaded into distal end portion 101 either within the first assembly member distal portion 24 or second assembly member distal portion 34, as shown in FIGS. 3A-D. Optionally treatment element 10 may be loaded into the distal end portion 101 of implant delivery system 50 through at least one or more inlet window 120, as shown in FIG. 4A-J.

Most preferably distal end portion 101 comprises at least one or more opening 122 to facilitate the delivery of treatment element 10. Optionally distal end openings 122 may be provided along at least one of first assembly member distal portion 24 or second assembly member distal portion 34. Optionally first assembly member distal end 24t or second assembly member distal end 34t are provided with an edge for example including but not limited to blunt, tapered, beveled, conical, tapered, pyramidal and pyramid like having a plurality of faces or the like.

Optionally at least one or more distal end openings provides for the delivery of at least one or more treatment element 10 at a target site 308, where the mode of delivery may vary to include at least one delivery manner for example including but not limited to lateral delivery, side delivery, end delivery, retractive delivery, pull back delivery, any combination thereof or the like.

Optionally at least one or more distal end openings may be provided with a cover 124 on at least one of first assembly member 20 or second assembly member 30. More preferably cover 124 may be controllably opened and closed. Most preferably cover 124 correspond to the shape of first assembly member distal end 24t or second assembly member distal end 34t (not shown) for example including but not limited to blunt, tapered, beveled, conical, tapered, pyramidal and pyramid like having a plurality of faces or the like.

Optionally distal end portion 101 provides for delivery of at least one or more treatment element by the relative movement of first assembly member distal end 24 with respect to second assembly member distal end 34 that may optionally be controlled with the proximal end portion 101.

FIGS. 3-5, 11 depict optional embodiments of the present invention for a system and method for a non-fluid implant delivery comprising a distal end portion 101 comprising a substantially blunt non sharp end 34t, 24t forming a implant delivery opening 122. The method of implant delivery with an optional implant delivery system having a blunt non sharp distal end 34t, as will be detailed below, is facilitated essentially in a two stage process. Optionally in a first stage, tissue within the target site is penetrated with a needle 112 or otherwise similar sharp edge preferably to form an implant delivery canal. Next in a second stage a treatment element is delivered to the target site, optionally with the assistance of a guide-shaft 116. The delivery method with the blunt non sharp distal end 34t, 24t therefore requires the sequential use of both a needle 112 and a shaft-guide 116 to a single delivery opening 122 that may only accept one at a time.

Optional embodiments of a non limiting configuration where entry to distal end 24t is limited to either needle 112 or shaft-guide 116, as described above by employing a treatment element loading window 120 that limits entry to only one of needle 112 or guide 116 in a one at a time manner via a single channel (FIG. 4D) or by a flexible separator or septum 130 (FIG. 4H).

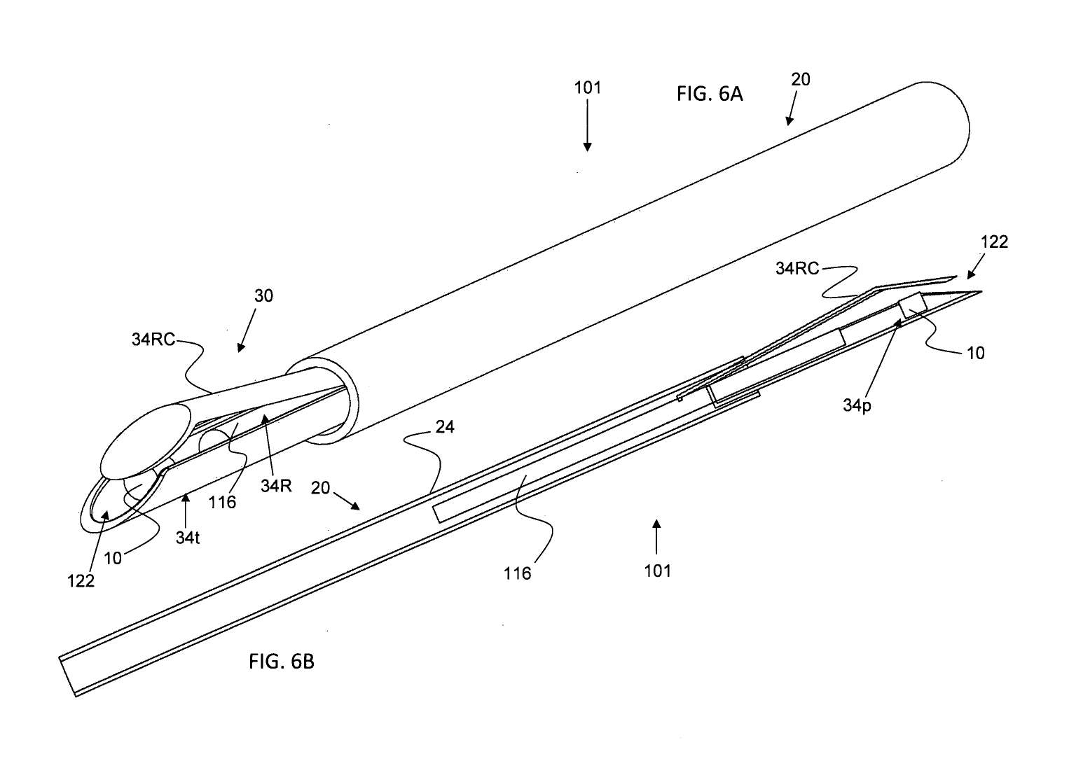

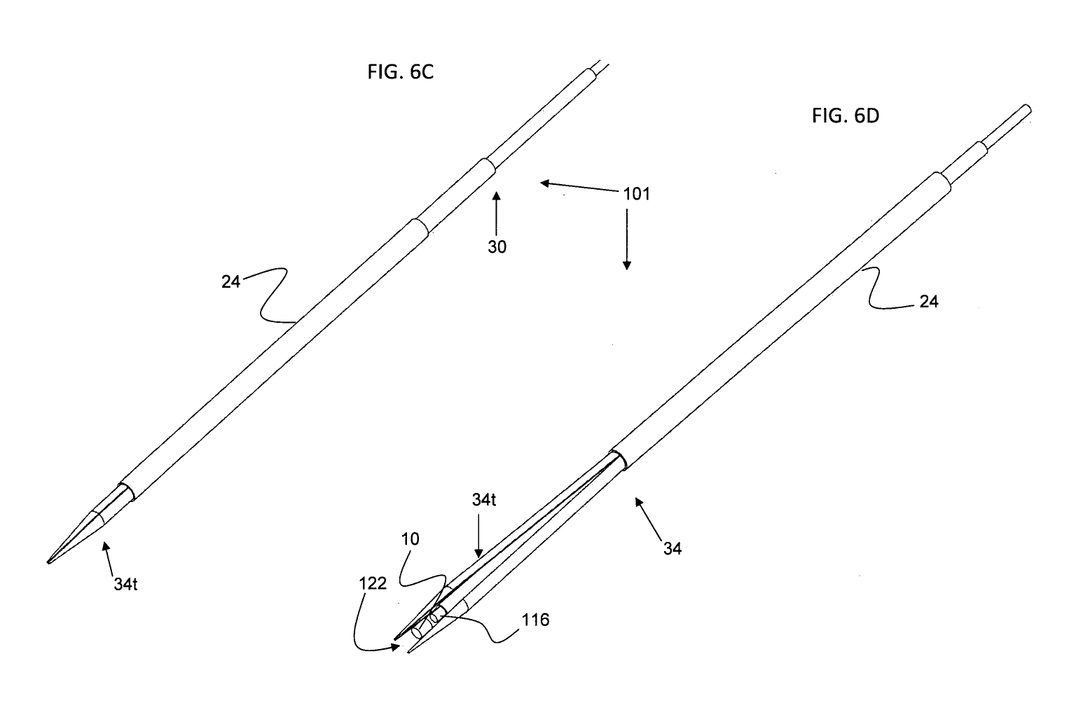

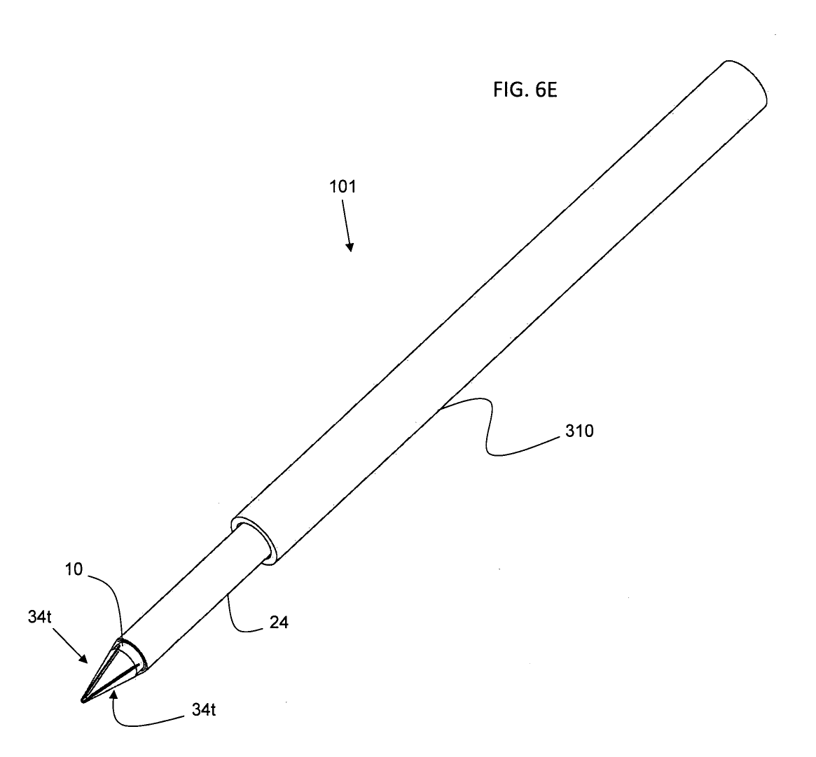

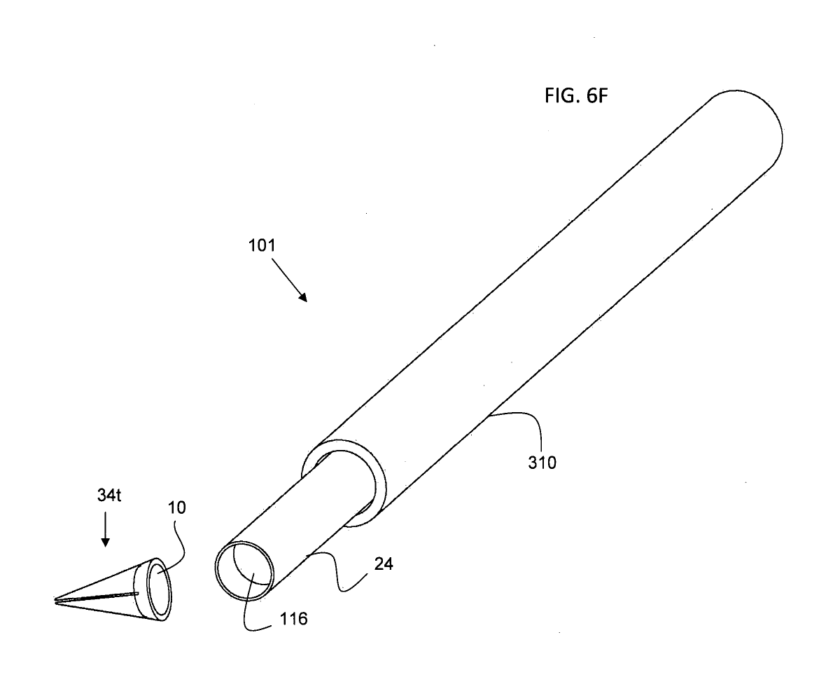

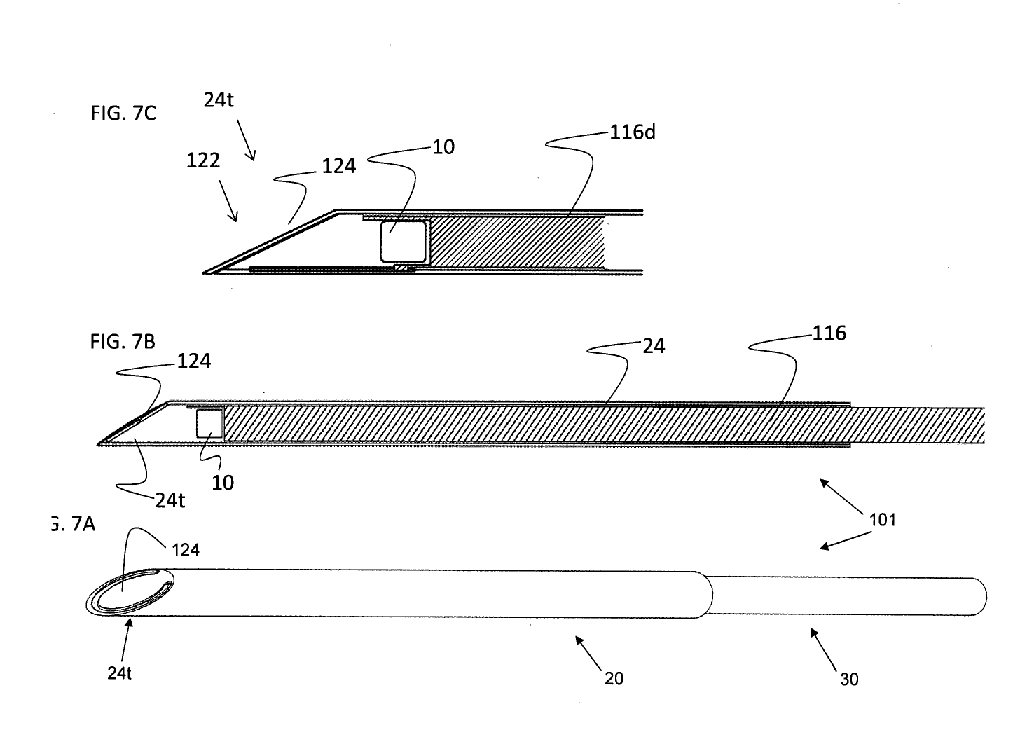

FIGS. 6-9 provide for integrating and essentially combining needle 112 into the distal end 34t, 24t of distal end portion 101 facilitating delivery without the use of a dedicated needle 112. As depicted in FIG. 6-9 sharp distal end 34t, 24t provided in various forms, beveled, conical, tapered facilitates the puncturing of a delivery canal within a target site 308 and delivery of treatment element 10 by going from a closed to an open configuration. Preferably the closed configuration is provided for penetrating and/or piercing into tissue preferably to form an implant delivery canal. Preferably the open configuration provides for the delivering treatment element 10 into the delivery canal. Most preferably distal end 34t,24t may be controllably opened, for example with the delivery guide-shaft distal tip adaptor 116d preferably facilitating opening cover 124, as depicted in FIG. 7C.

Referring now to FIG. 3A-D show schematic illustrations of an optional distal end portion 101 according to the present invention provided for the delivery of at least one or more treatment element 10 characterized in that at least one or more treatment element 10 may be preloaded within second assembly member 30 within second assembly member distal end 34 as shown in FIG. 3B. Optionally treatment element 10 may be preloaded within first assembly member 20 within first assembly member distal end 24. Optionally treatment element 10 is associated with a treatment element housing 10h for maintaining treatment element 10 until delivery.

Optionally treatment element 10 may be loaded into at least one or more portion of distal end portion 101. Optionally once loaded treatment element 10 is disposed within a housing 10h.

FIGS. 3A-D depict a non limiting optional embodiment where first assembly member distal end 24 or second assembly member distal end 34 is provided with a blunt substantially cylindrically shaped end 24t, 34t forming a delivery opening 122 for delivering at least one or more treatment element, optionally and preferably providing for an end delivery manner through delivery opening 122.

FIG. 3A provides a perspective view of an optional non limiting distal end portion 101 distal portion comprising first assembly member 24, and distal portion of second assembly member 34. First assembly member distal end 24 comprises a blunt substantially cylindrically hollow shaped end 24t forming a delivery opening 122 for delivering at least one or more treatment element 10. Second assembly member distal portion 34 comprises a needle 112 optionally and preferably, which can be advanced longitudinally ahead of the blunt tip 24t, provided for forming a delivery canal and treatment element delivery shaft-guide 116 for advancing and or urging at treatment element 10 distally toward delivery opening 122, optionally and preferably limited to stop slightly behind of the blunt tip 24t

FIG. 3B provides a longitudinal sectional view of the distal end portion 101 depicted in FIG. 3A providing an luminal view of the preloaded distal end portion 101, showing treatment element 10, delivery shaft-guide 116 that optionally is a non-hollow needle. FIG. 3B shows an optional configuration wherein treatment element 10 is preloaded within distal portion of second assembly member distal portion 34. Optionally delivery is provided by advancing treatment element 10

Optionally and preferably the outer diameters of the shaft-guide 116 and of needle 112 are essentially the same, and the inner diameter of member housing 100 is slightly larger than the combined inner diameters of guide 116 and needle 112, and the inner diameter of distal end 24t is approximately half of inner diameter of housing 100, so that the inner diameter of 24t is slightly larger but almost the same of the outer diameters of shaft-guide 116 and of needle 112. During implant delivery needle 112 may be advanced first, and then withdrawn backward to enable the advancement of shaft guide 116. Optionally delivery is provided by advancing treatment element 10 with delivery shaft-guide 116 e treatment element 10 toward pre-implantation site 24p preferably within the lumen of distal end 24t and immediately preceding opening 122, where most preferably treatment element 10 is placed just prior to implant delivery within the delivery canal of target site 308. Most preferably delivery is provided by advancing treatment element 10 with shaft-guide near the distal end opening 122 where implant 10 does not penetrate the delivery canal (not shown) through opening 122 rather distal end portion 101 may optionally be retracted proximally while implant 10 is dropped, therein placed within the delivery canal (not shown) at the same position, in relative to the body, as acquired in pre-implantation site 24p.

Optionally, after complete withdrawing of shaft-guide 116 but prior withdrawing the entire delivery system the operator in addition to the implant delivery can stream fluid through the system, optionally through an opening (not shown) at the proximal portion 150.

FIG. 3C provides a perspective view of an optional non limiting example of a distal end portion 101 while FIG. 3D provides a longitudinal section depicts an intra luminal view of distal end portion 101 of FIG. 3C. Distal end portion 101 of FIG. 3C comprising first assembly distal member 24 and second assembly member distal end 34.

Second assembly member distal end 34t comprises a blunt substantially cylindrically shaped tip forming a delivery opening 122 for delivering at least one or more treatment element 10. First assembly distal portion 24 comprises treatment element 10, needle 112 and an adaptor 118 in the form of a needle to shaft-guide adaptor. Optionally first assembly member 20 may be provided with a delivery shaft-guide 116 and an adaptor 118 in the form of shaft-guide to needle adaptor. Optionally, adaptor 118 provides for using a single device for two functions for example puncturing or piercing a delivery access route 306 (described in FIG. 1A-C) with the needle and urging or advancing treatment element 10 toward pre-implantation site 34p and then dropped into target site 308

Distal end portion 101 of FIG. 3C optionally provides for the delivery of at least one or more preloaded implant 10 into target site 308 (not shown) by forming a delivery access route 306 (not shown) with needle 112 disposed in first assembly member 20 by advancing the needle 112 distally through distal opening 122 disposed in second assembly member distal end 34, FIG. 3E, then retracting needle 112 proximally into first assembly 20,wherein adaptor 118 is displaced into the lumen of distal end 34 with an adaptor displacer 119 FIG. 3F providing for attaching needle 112 to adaptor 118, as shown in FIG. 3F, therein optionally and preferably converting needle 112 to a shaft-guide 116 and then advancing implant 10 distally toward opening 122 where optionally implant 10 may be advanced distally past opening 122 or more preferably distal end 34t may be retracted backwards placing implant 10 within delivery canal of target site 308 at a fixed position in relative to the body.

FIGS. 4A-J are schematic illustrations of optional non limiting embodiments of distal end portion 101 of the apparatus according to the present invention provided for the delivery of at least one or more treatment element 10 that is most preferably loaded through at least one or more treatment element inlet loading window 120. Optionally at least one or more treatment elementelement inlet loading windows 120 may optionally be disposed on at least one, both or spanning at least a portion of both of the first assembly member 20 or second assembly member 30. Optionally at least one or more inlet loading window 120 is disposed on the first assembly member distal portion 24.

Optionally distal end portion 101 comprising at least one or more treatment element inlet window 120 optionally and preferably further comprises a controllable holder 130 for maintaining implant 10 within delivery inlet window 120 for example including but not limited to cover (depicted in FIG. 4G), door 128 (shown in FIG. 4F), auxiliary device, separator 130, stopper (shown in FIGS. 4B, 4D, 4H, 4I), any combination thereof, or the like.

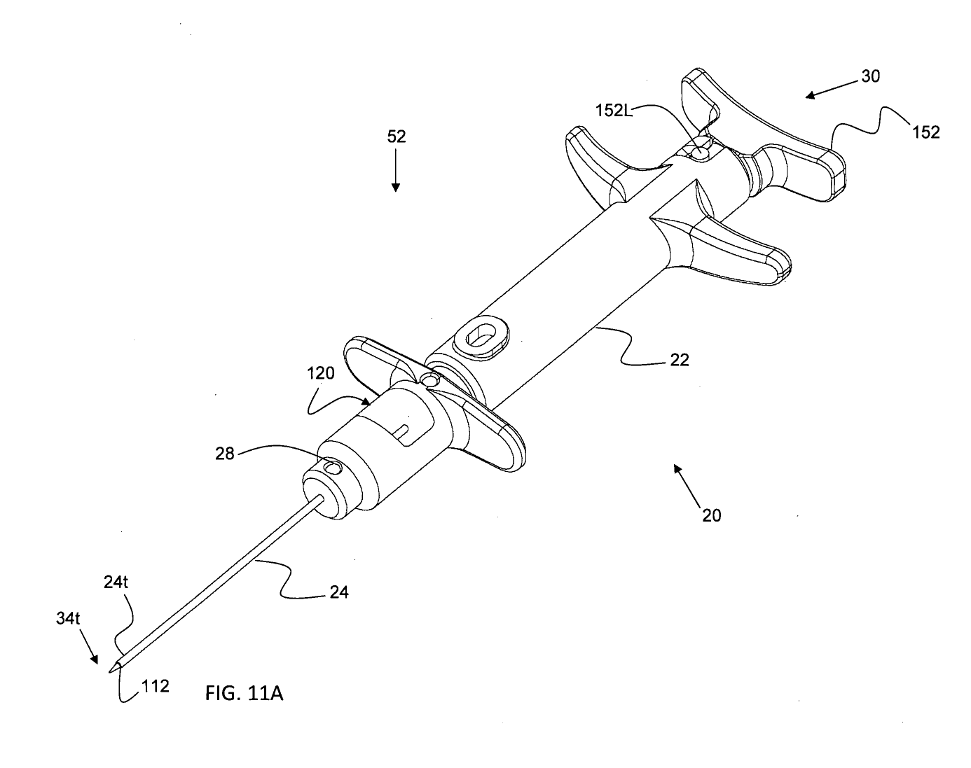

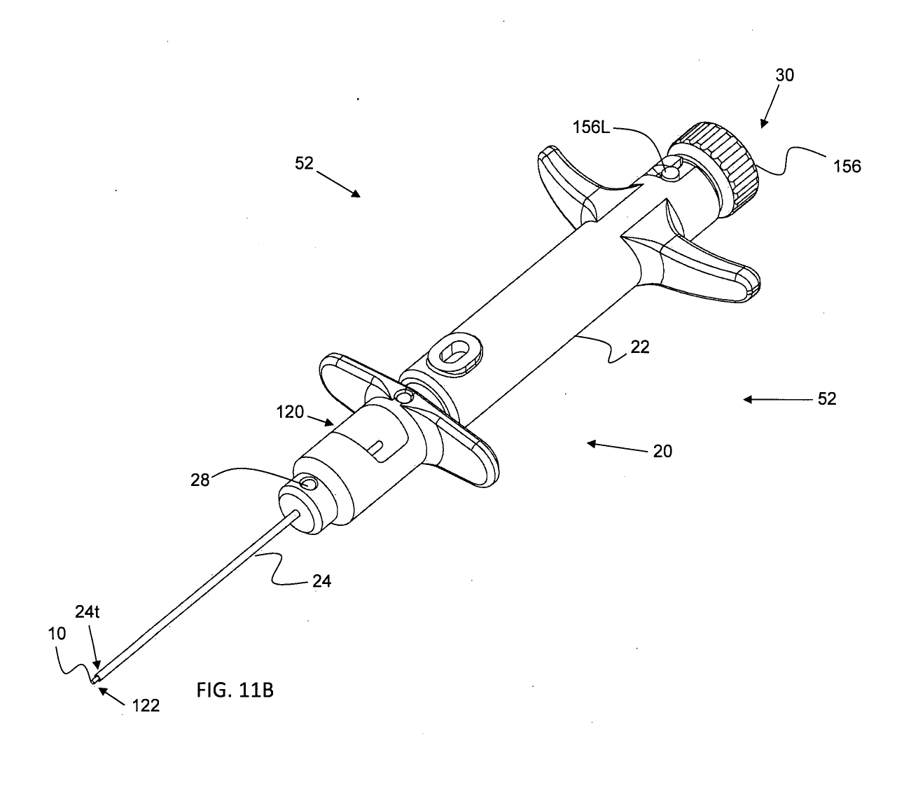

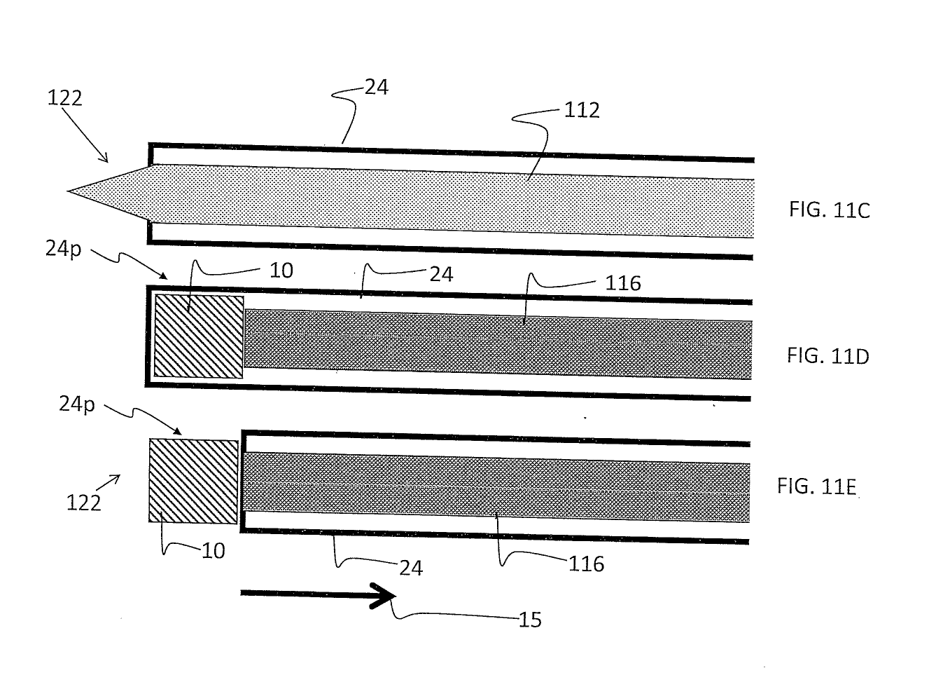

Optionally inlet loading window 120 may be provided in optional shapes so as to provide a unidirectional window allowing a treatment element in but not out of the same window, optionally the window 120 may be trapeze conically shaped, as shown in FIG. 11A-E.

Optionally inlet window 120 may be configured according to at least one or more dimensional parameters associated with the treatment element 10.

FIG. 4A depict a non limiting optional embodiment of distal portion 101 where second assembly member distal end 34t is provided with a blunt substantially cylindrically shaped forming a delivery opening 122 for delivering at least one or more treatment element, optionally and preferably providing for an end delivery and/or retractive delivery manner. Optionally distal end 34t may be displaced backwards into first assembly member distal portion 24. Preferably at least one or more treatment element 10 is loaded into distal end portion 101 through inlet window 120 prior to delivery.

Optionally distal end portion 101 of FIGS. 4A-J may be utilized with an auxiliary device for example including but not limited to a trocar, guiding catheter, catheter, endoscope, endoscope with working channels, endoscope with ultrasound probe, introducer, stepper, port, sleeve, or the like, to facilitate implant delivery according to the present invention.

Optionally when an auxiliary device is associated with distal end portion 101 depicted in FIGS. 4A-J at least one or more treatment element 10 are loaded into inlet loading window 120, prior to associating distal end portion 101 with an auxiliary device, for example an endoscope having a working channel 316 or the lumen of a catheter (not shown) or trocar (not shown), as described in FIGS. 1A-B),

Optionally delivery is provided by advancing treatment element 10 past opening 122 with delivery shaft-guide 116. Most preferably delivery is provided by advancing treatment element 10 with shaft-guide 116 near the distal end opening 122 optionally guide 116 is stopped proximally to the opening 122 at distance about the length of the element 10 where implant 10 does not penetrate the delivery canal (not shown) through opening 122, rather, distal end portion 101 and/or distal end 34t are retracted proximally while implant 10 is placed within the delivery canal (not shown) in a fixed position in relative to the body.

FIG. 4D shows an optional configuration wherein treatment element 10 is loaded through inlet loading window 120 disposed on the first assembly member distal portion 24 and is temporarily held on top of needle 112 disposed in the second assembly member distal portion 34. Optionally delivery is initiated with the formation of a delivery canal with needle 112 at the target site 308. Next needle 112 is retracted backwards into the first assembly member 20 proximally passing inlet loading window 120 therein releasing treatment element 10 into the lumen of first assembly member 20. Next preferably a shaft-guide 116 disposed in second assembly member distal portion 34 is advanced distally toward treatment element 10. Optionally, delivery is provided by advancing treatment element 10 past opening 122 with delivery shaft-guide 116, as may be provided by distal end portion 101 of FIGS. 4D-G, 4I-J. Most preferably delivery is provided by advancing treatment element 10 with shaft-guide 116 near the distal end opening 122 where implant 10 does not penetrate the delivery canal (not shown) through opening 122 and preferably stopping at pre-implantation site 24p, where distal end portion 101 is retracted proximally (backward) while implant 10 is urged distally therein placed within the delivery canal (not shown), as may be provided by distal end portion 101 of FIGS. 4C-D, 4E-F, 4I-J. Optionally, distal end 34t may be retracted proximally therein placing implant 10 within delivery canal of target site 308, as may be provided with the distal end portion 101 of FIGS. 4A-B, 4G-H.

Referring to FIGS. 41 and 4J showing an optional depiction of a distal end portion 101 comprising a plurality of inlet loading windows 220. Optionally inlet loading windows 220 may provide for sequentially delivering a plurality of treatment element 10. Optionally different treatment elements may be disposed in individual inlet loading windows 120 forming inlet loading window 220, therein optionally providing for the delivery of different combination of treatment element within a target site 308. Optionally and preferably inlet loading window 220 is provided such that sequential implant delivery is possible where individual treatment element 10 do not interact or come into contact with one another.

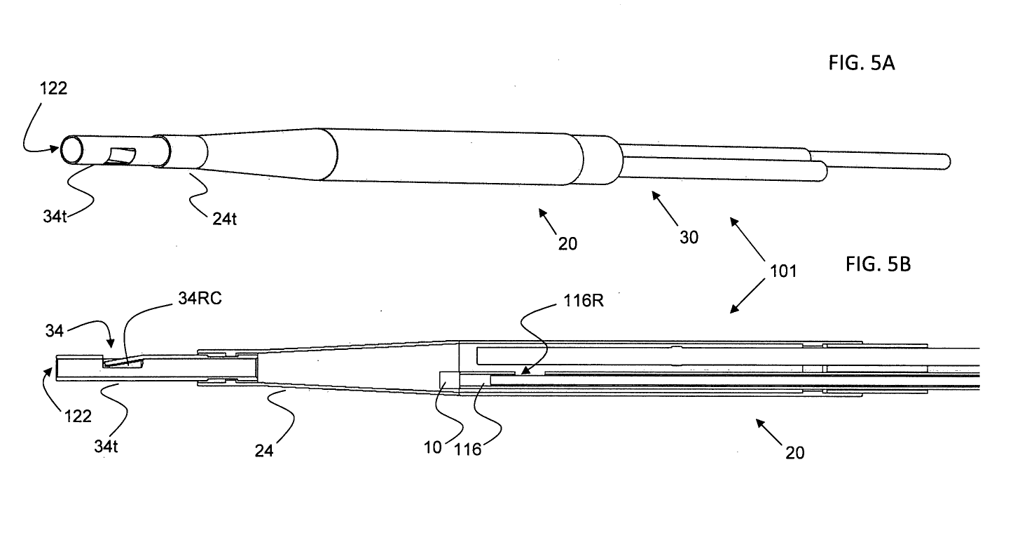

FIGS. 5A-B are schematic illustrations of optional non limiting embodiments of a distal end portion 101 of the system 50, adapted to minimize the movement of system 50, by retracting only a portion of the distal portion 101 of system 50. According to the present invention provided for the delivery of at least one treatment element 10 via a retractive and/or pull out manipulation of the distal end 34t or 24t according to an optional embodiment of the present invention.

FIG. 5A provides a perspective view of distal end portion 101 wherein first assembly member 20 comprising a second assembly member 30 disposed concentrically with first assembly member 20 wherein second assembly member distal end 34t extend distally to first assembly member distal end 24t. Optionally second assembly member distal end 34t is provided with a blunt substantially cylindrically shaped forming a delivery opening 122 for delivering at least one or more treatment element. Optionally distal end 34t may be provided with an edge for example including but not limited to blunt, tapered, beveled, conical, tapered, pyramidal and pyramid like having a plurality of faces. Optionally distal end 34t may further comprise a covering (not shown).

Optionally and preferably distal end 34t provides for end delivery and/or retractive implant delivery manner. Most preferably distal end 34t may be displaced backwards into first assembly member distal end 24. Preferably at least one or more treatment element 10 may be delivered with the retractive delivery method as described above wherein distal tip 34 is retracted into first assembly member distal tip 24 therein placing treatment element 10 within the delivery canal of target site 308. Most preferably retraction of distal end 34t into first assembly distal portion 24 facilitates delivery of treatment element 10 without requiring the displacement of distal end assembly portion 101.

FIG. 5B depicts distal end 34t comprising at least one or more recess 34r comprising at least one coupling member, for example in the form of a recess cover 34rc provided for coupling with a corresponding coupling member optionally disposed in first assembly member 20 or second assembly member 30, more preferably disposed within a second assembly member, for example shaft-guide 116. Most preferably shaft-guide 116 comprises a corresponding coupling member to distal recess cover 34rc, for example in the form of recess 116r. Optionally and preferably recess cover 34rc and recess 116r may associate with one another once shaft-guide 116 is displaced distally toward opening 122 providing for the coupling of recess 116r and recess cover 34rc, therein coupling shaft guide 116 with distal end 34t. Distal end 34t may then moved backward into the lumen of first assembly member 20 at distal portion 24 with the backward displacement of shaft-guide 116 intraluminally into first assembly member distal portion 24.

FIGS. 6A-F are schematic illustrations of optional non limiting embodiments of a distal end portion 101 of the system 50 according to the present invention provided for the delivery of at least one treatment element 10 by manipulating distal end 34t or 24t. FIGS. 6A-F provide schematic illustration of the system according to an optional non limiting embodiment of the present invention wherein needle 112 is integrated within the external portion comprises a sharp end provided for penetrating, piercing a target site for the delivery. Most preferably the sharp edge is provided for reducing the number of manipulations and/or movement required for penetrating the target site with an auxiliary device for example in the form of a needle 112. Moreover the sharp edge may also enable effective coating of the distal part 24 that shields the system at the period prior to implant delivery and during the first stages of implant delivery for example initial penetration, until the final delivery of the implant and/or treatment element.

Referring now to FIGS. 6A-B showing schematic illustrations of an optional non limiting embodiment of distal end portion 101 providing for an end delivery and/or pull back delivery manner as previously described. FIG. 6A shows a perspective view while FIG. 6B provides a longitudinal cross sectional intraluminal view. Distal end portion 101 of FIGS. 6A-B comprise a first assembly member 20, and a second assembly member 30 that comprises substantially beveled distal end 34t, a distal tip recess cover 34rc, delivery shaft-guide 116. Optionally and preferably distal end 34 comprises an opening along its length forming a lengthwise recess 34r therein defining a delivery opening 122. Preferably lengthwise recess 34r is provided with a corresponding 34rc therein providing both an open and closed formation for distal end 34t of FIG. 6A-B. Optionally and preferably shaft guide 116 may be coupled with distal tip recess cover 34rc to controllably provide a cover for distal tip 34 having a beveled tip 34t and recess 34r. Most preferably the proximal displacement of shaft-guide 116 into first assembly member 20 provides a closed formation of distal end 34t while distal displacement of shaft-guide 116 provides an open formation of distal end 34t.

Optionally the retractive delivery provided for distal end portion 101 of FIG. 6A-B is adapted from that previous described as distal end 34t having a beveled shape provides for creating the delivery canal at target site 308. Once the delivery canal is created distal displacement of shaft-guide 116 forms delivery opening 122 as treatment element 10 is gently advanced towards the delivery canal and optionally stopped at the opening 122 such that treatment element 10 does not cross distal end 34t and is placed at the delivery canal by retracting backwards distal end portion 101 or distal portion 34.

Referring now to FIGS. 6C-D showing schematic illustrations of an optional non limiting embodiment of distal end portion 101, where FIG. 6C depicts a perspective view of a closed formation while FIG. 6D shows the open formation of distal end portion 101. Optionally distal end portion of FIG. 6C-D comprises a first assembly member 20 concentrically associated with second assembly member 30. Optionally second assembly member distal end 34t comprises at least two halves providing for an open (FIG. 6D) and a closed (FIG. 6C) formations. Optionally second assembly member distal end 34t is provided with a tapered edge or a semi-conical edge that may optionally and preferably provide for gaining access to a target site 308 and forming a delivery route 316. Optionally distal end 34t comprises at least two halves that may further concentrically comprise at least one or more treatment element 10 and a delivery shaft-guide 116. Optionally the change from a closed formation to an open formation of distal end 34t is controllable by manipulating first assembly member 20 proximally while the second assembly member 30 is displaced distally, forming delivery opening 122. Optionally distal end 34t are provided in the form resembling forceps that may form an open or closed formation.

Optionally and preferably once opened a delivery opening 122 is formed providing for delivery. Implant delivery of at least one or more treatment element 10 may be provided in the open formation (FIG. 6D) where the treatment element 10 is placed into the delivery canal (not shown). Optionally delivery may be facilitated with shaft-guide 116 gently urging treatment element 10 into the delivery site, optionally in pull back retractional manner, or an end delivery manner, as previously described.

Referring now to FIGS. 6E-F showing schematic illustrations of an optional non limiting embodiment of distal end portion 101, where FIG. 6E depicts a perspective view of a closed formation while FIG. 6F shows a partially exploded view of FIG. 6E. Second assembly member distal end 34t may optionally and preferably be provided as an independent member that may be coupled to the remaining assembly. Most preferably distal end 34t is loaded with at least one or more treatment element 10 that may be placed within the target zone 308 with the aid of shaft-guide 116. Preferably distal end 34t is associated with the assembly of distal end portion 101 prior to use. Optionally the system described herein is associated for example with an auxiliary device for example including but not limited to a catheter, endoscope 310, trocar or the like. Distal end 34t may be provided in a plurality of forms provided in an open configuration (not shown) and a closed configuration. Optionally distal end 34t may be opened by the forward (distal) movement of shaft-guide 116 toward the delivery canal therein urging treatment element 10 toward the target site 308 while opening distal end 34t.

Although distal end 34t is depicted in the form of a conical claw like formation having a plurality of faces it may for be provided in a plurality of optional shapes for example including but not limited to conical, pyramidal and pyramid like having a plurality of faces, or the like closed structure with at least two or more faces that may be controllably opened or closed. Most preferably distal end 34t is opened to create a treatment element delivery opening 122. Optionally distal end 34t may be provided with a sharp edge for facilitating the creating of a treatment element delivery canal within a treatment zone.

Referring now to FIGS. 7A-C are schematic illustrations of an optional distal end portion 101 of system 50. FIG. 7A provides a perspective view of an optional non limiting embodiment of a distal end portion 101 comprising a first assembly member 20 and a second assembly member 30 that are preferably disposed concentrically to one another. FIG. 7B provides a longitudinal section of FIG. 7A, and FIG. 7C provides a close up view of distal end 101d of FIG. 7C. First assembly member housing 20 preferably comprises a distal end 24t having a sharp end, preferably a beveled end, optionally and preferably provided for penetrating target site 308 therein forming a delivery route 306. Optionally and preferably distal end tip 24t may further comprise a cover 124 covering to cover distal tip 24t providing a controllable distal end that may controllably be opened or closed with the distal tip of guide-shaft 116d such that when guide shaft 116 it is moved forward cover 124 is lifted revealing delivery opening 122 (not shown) for the delivery of at least one or more treatment elements 10.

Optionally delivery guide-shaft distal tip 116d may be provided in the form of an adaptor that may optionally be associated with optional auxiliary tools and moved with the associated auxiliary tool, for example by air pressure, vacuum, or manual manipulation.

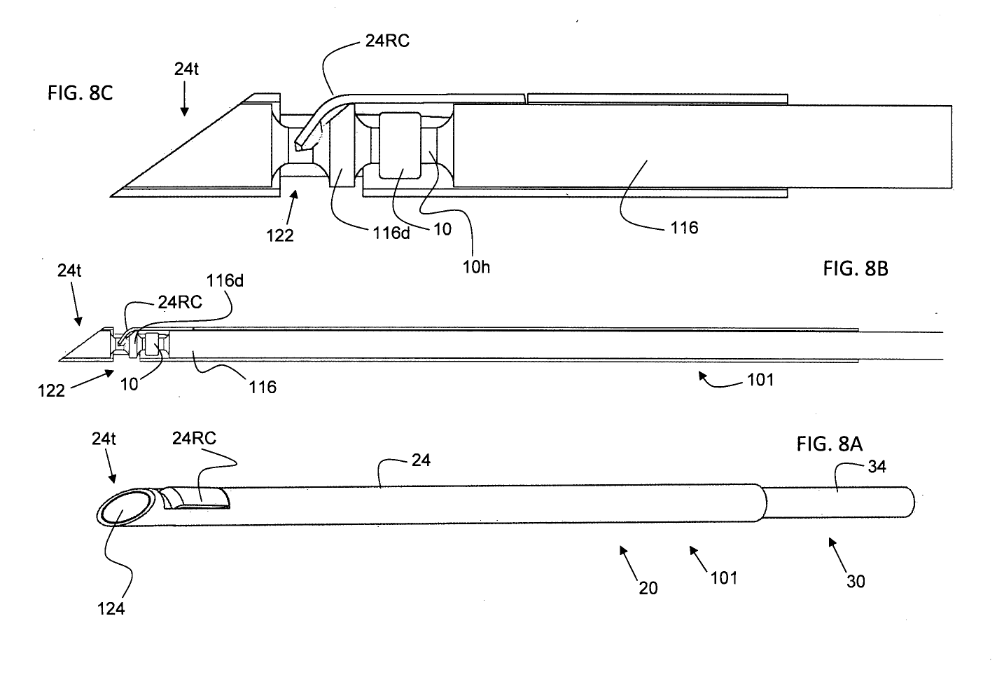

Referring now to FIGS. 8A-C showing schematic illustrations of a non limiting optional embodiment of distal end portion 101 optionally adapted to side delivery of element 10. FIG. 8A provides a perspective view of an optional non limiting embodiment of a distal end portion 101 comprising a first assembly member 20 and a second assembly member 30 that are optionally disposed concentrically to one another. FIG. 8B provides shows a longitudinal cross section of FIG. 8A while FIG. 8C provides a close up view of distal end portion 101.

First assembly member housing 20 preferably comprises a distal end 24t at the distal end of distal portion 24 having a sharp end optionally and preferably provided for penetrating target site 308 therein forming a delivery route 306. Optionally and preferably distal end 24t comprises a recess 24r and recess cover 24rc for engaging second assembly portion member distal end 34t. Optionally and preferably distal end 24t may be provided as a non-hollow needle, optionally distal end 24t may be provided as a hollow needle with an integrated distal end cover 124. Optionally a distal end 24t may be coated with a biocompatible polymeric seal for example including PLGA, PLA, PCL or the like biocompatible sealant.

Second assembly member preferably comprises at least one treatment element 10 (FIG. 8B), second assembly member distal end 34t and shaft-guide 116. Preferably shaft-guide 116 is provided with a distal end 34t provided for displacing cover 24rc and at least one or more treatment element 10 proximally to distal end tip 34t. Optionally and preferably once distal cover 24rc is displaced upright as distal end tip 34t is displaced distally it brings treatment element in alignment with side delivery opening 122 provided for the side or lateral delivery of at least one or more treatment element 10. Preferably side delivery of treatment element 10 is provided for by bringing treatment element 10 into side delivery opening 122 is effected once shaft-guide 116 is further displaced distally toward first assembly member distal end 24t such that distal end 34t moves distally past cover 24rc therein releasing cover 24rc intraluminally urging treatment element 10 into the target site through side delivery opening 122.

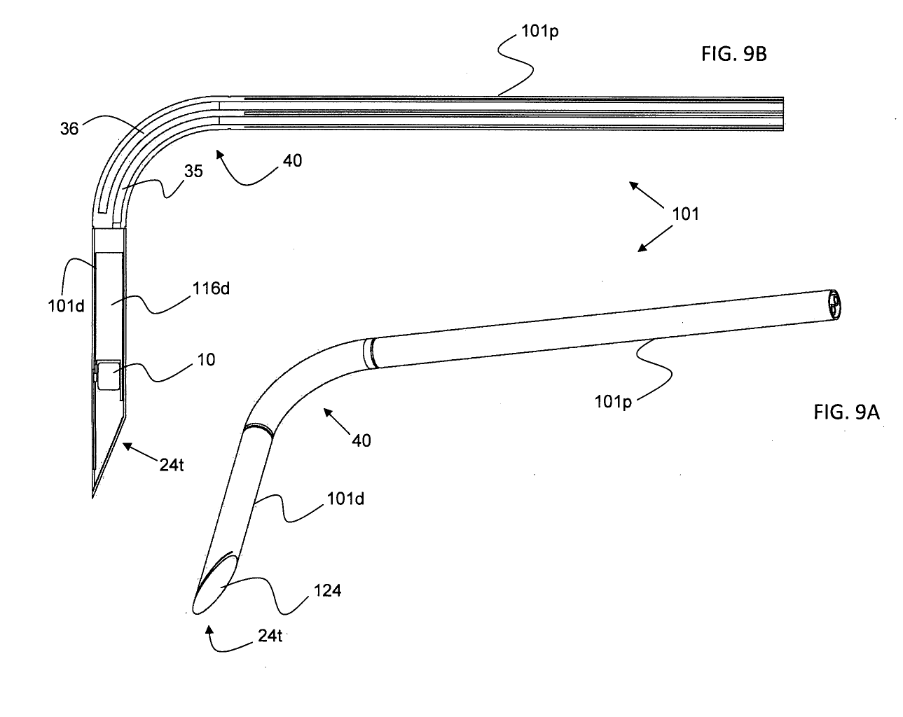

FIGS. 9A-B provide an optional schematic illustration of a non limiting embodiment of a system according to the present invention wherein the system provides for angular and/or radial delivery of at least one or more treatment element. FIG. 9A provides perspective view of an optional distal end portion 101 adapted for angular and/or radial delivery. Distal end portion 101 may be provided in the same form much like other distal end portion 101 previously described for example in FIGS. 3-8 however further comprising a flexible mediating assembly 40 disposed essentially between the proximal portion 101p and distal end portion 101d. Optionally and preferably mediating assembly 40 provided distal end portion with a controllable angle from about 0 degrees to about 120 degrees with respect to 101p.

FIG. 9B provides a longitudinal section of FIG. 9A wherein implant treatment element 10 is associated with delivery guide-shaft distal tip adaptor 116d. Optionally curvature of flexible mediating assembly 40 is provided for with pulling wires, air compression, vacuum, hydraulic compression, pre-shaped balloon, asymmetric threading or the like, for example as utilized in maneuvering an endoscope.

FIG. 9B depicts an optional embodiment where curvature of mediating assembly 40 is provided with at least one of first member 35 or a second member 36. Optionally first and second members 35, 36 may function concertedly to bring about the required curvature of mediating member 40. Optionally mediating assembly 40 may assume an angle from about 0 degrees to about 120 degrees.

Optionally at least one of member 35 or member 36 may further facilitate the delivery of implant 10 by urge guide-shaft dist tip adaptor 116d toward the delivery canal to facilitate the delivery of treatment element 10 through distal tip 24t by lifting cover 124 with adaptor 116d.

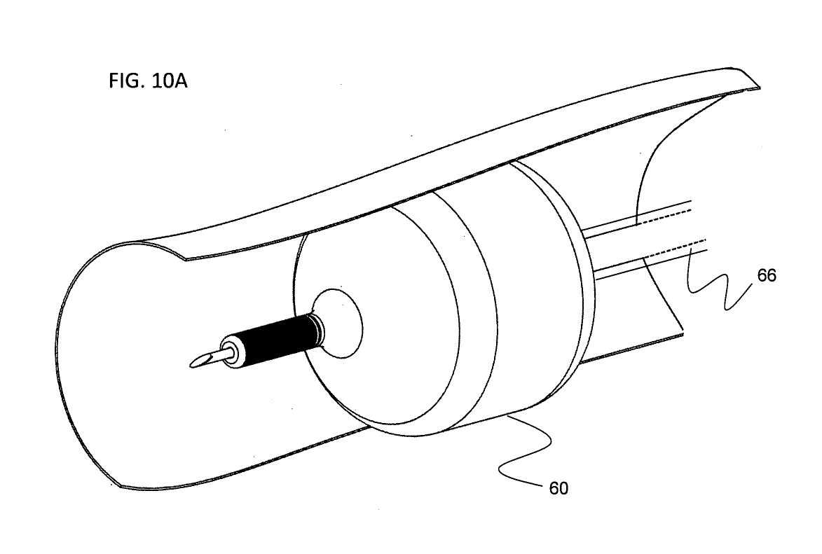

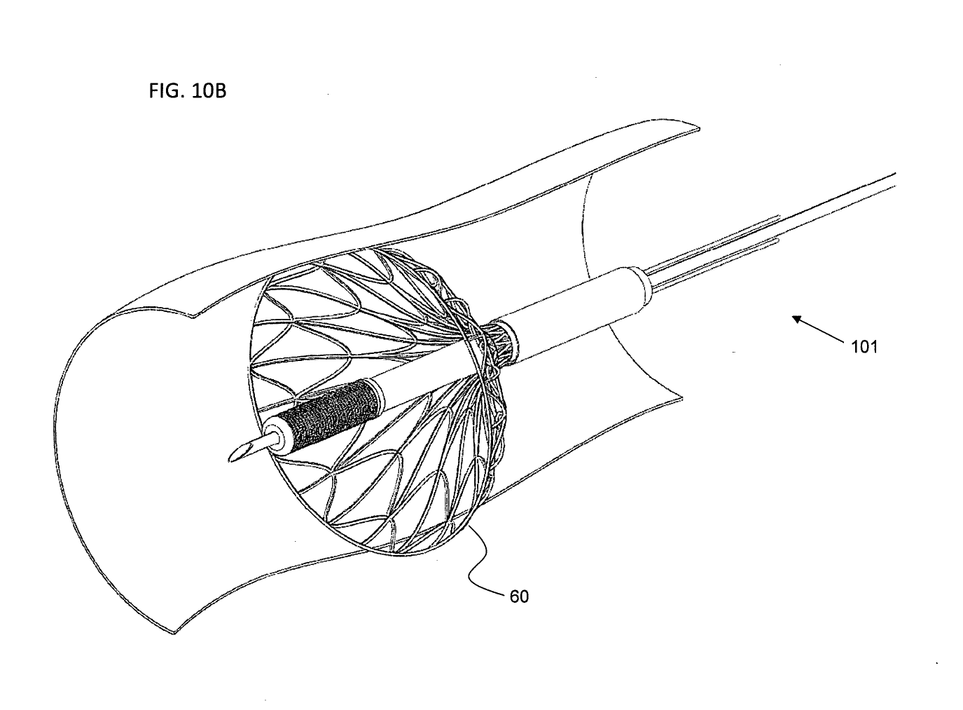

Optionally a fluid may be delivered (in addition to the delivery of the implant) in the vicinity of and/or the delivery route to the target site through at least one of first or second members 35, 36 following implantation of treatment element 10. FIG. 10A-B provide a schematic illustration of optional non limiting embodiments of an optional delivery system according to the present invention comprising a stabilizing device 60 that may optionally be provided in a plurality of forms for example including but not limited to balloon, net, filter, mesh, metallic mesh, ring, anchors or the like. An optionally stabilizing device 60 in the form of a semi-compliant balloon is schematically depicted in FIG. 10A. Balloon 60 is provided to stabilize and provide a counter balance for implant delivery system distal end 101 during the delivery of at least one or more treatment element. Optionally balloon 60 is controllably expanded near a delivery site to stabilize implant delivery. Optionally balloon 60 is inflated using balloon inflating tube 66 in similar manner to known medical balloons having an inner optionally cylindrical hollow canal. In other applications, for example balloon angioplasty and balloon expandable stents, such a cannal typically is used for guide wire, while in this invention it may be used for the delivery system described in previous figures. FIG. 10A depicts the expanded balloon 60 near a delivery site (not shown) that may be optionally located distally and/or above mediating member 40.

FIG. 10C provides an optional embodiment of stabilizing device 60 provided in the form of a controllable metallic mesh that may be expanded prior and during delivery of at least one or more treatment element 10 (not shown) and retracted following delivery.

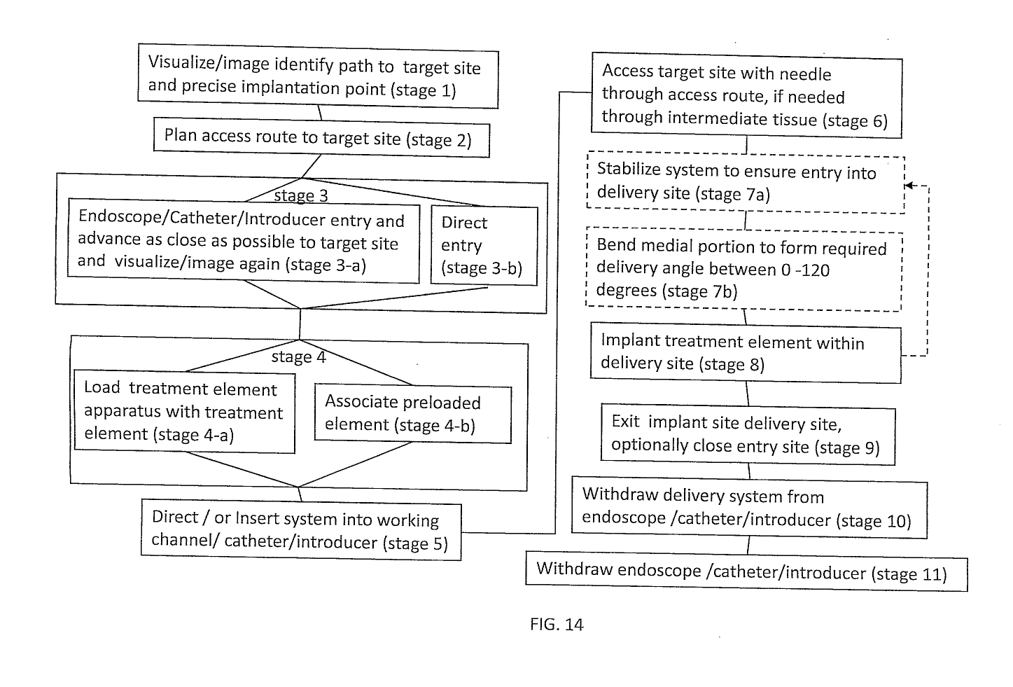

FIG. 11A-E provide a schematic illustration of an optional non limiting embodiment of the present invention wherein the delivery system is provided in the form resembling a non-flexible syringe for the delivery of at least one or more treatment elements, as described in FIGS. 1C and 14.

Optionally the delivery procedures relating to embodiments illustrated in FIG. 11C-E are performed directly without an additional channeling device, for example for topical delivery to portion of the body for example organs including but not limited to eye, skin, vagina, rectum and the like. Optionally the delivery procedures relating to embodiments illustrated in FIG. 11A-E are performed directly with an additional channeling device, for example an introducer, including introducer called “coaxial” and sheath introducer, which optionally are stiff.

FIG. 11A provides a perspective view of an optional non limiting system 52 comprising a first assembly member 20 and a second assembly member 30 wherein second assembly member 30 is associated with first assembly member 20. First assembly member 20 comprises a proximal end 22 and a distal end 24.

First assembly member distal end portion 24 preferably in the form of a continuous shaft comprises a blunt substantially cylindrically shaped tip 24t forming a delivery opening 122 for delivering at least one or more treatment element 10 (not shown). First assembly member 20 further comprises a treatment element inlet loading window 120 for loading at least one or more treatment elements into system 52, optionally prior to, or during delivery process. For example in case of more than a single delivery element 10 (not shown) planned to be delivered the first element could be preloaded and the rest elements can be sequentially loaded during the delivery process.

Second assembly member 30 comprises proximal end 32 and distal end 34t. Preferably a plurality of optional second assemblies 30 may be utilized with first assembly member 20 for example a second assembly member comprising a conical or tapered or pyramid or beveled tip or a blunt substantially conical end (FIG. 11B). Optionally second assembly member 30 comprising a sharp tip for example a conical or tapered or pyramid or beveled tip 34t (FIG. 11A) is provided for forming a delivery canal while a second assembly member comprising a blunt tip (FIG. 11B) provides for urging at least one or more treatment element distally toward the delivery canal.

Preferably a plurality of optional second assemblies 30, for example guide 116 and needle 112, may be utilized with first assembly member 20. For example, a second assembly member comprising a conical or tapered or pyramid or beveled tip for example in the form of a needle shaft 112, FIG. 11A. For example a second assembly member in the form of a blunt substantially conical end (not shown) 116, FIG. 11B.

Optionally second assembly member 112 comprising a sharp tip for example a conical or tapered or pyramid or beveled tip 34t (FIG. 11A) is provided for forming a delivery canal and preferably controlled with proximal controller 152; while a second assembly member 116 comprising a blunt tip (not shown) provides for urging at least one or more treatment element distally toward the delivery canal with controller 156, that optionally and preferably is provided with a controllable shaft-guide lock 1561, preferably provided to limit the length shaft-guide 116 may be advanced thought shaft 24. Most preferably shaft-guide lock 156 provides for limiting the penetration of shaft-guide 116 through shaft 24 such that the distal end 34t (not show) of shaft-guide 116 may only reach the pre-implantation site 24p ensuring that treatment element 10 remains in a fixed location relative to the body during the implant delivery.

Optionally system 52 may be provided with a shaft along first assembly distal end 24 comprising a controllable length to adjust to a particular implantation site, optionally shaft lock 28.

FIG. 11B shows system 52 where second assembly member distal tip is provided with a distal blunt 34 for facilitating delivery of at least one or more treatment elements 10 (not shown).

An optional and preferable method of delivery with system 52 of FIGS. 11A-B is depicted in FIGS. 11C-E. Optionally delivery is provided by advancing treatment element 10 just proximately to opening 122 with a blunt second assembly member shaft guide 116 disposed in distal end 34, as shown in FIG. 11C-D then retracting distal end 34t to drop element 10 in the delivery canal. Optionally retractive delivery is provided by advancing treatment element 10 toward and most preferably not past opening 122 with blunt second assembly member guide 116 at distal tip 34t as shown in FIG. 11D-E.

As depicted in FIG. 11C, delivery is optionally and preferably initiated with the formation of a delivery canal with needle 112 preferably comprising a sharp edge for penetrating target site 308 for example sharp distal end 34t that is preferably non-hollow, preferably comprising a handle tailored to pull out second assembly member, acting like a needle 112 or the like sharp penetrating assembly as shown in FIGS. 11A, 11C where implant delivery is made within the target site 308. Next sharp distal end 34t of needle 112 is retracted proximally out of first assembly member distal end shaft 24 and replaced with a second assembly member, preferably in the form of shaft guide 116, having a blunt end 34t as depicted in FIGS. 11B, D-E, preferably comprises a handle tailored to push and fix. Preferably a treatment element 10 may then be loaded through treatment element inlet window 120 and advanced distally with shaft guide 116 comprising blunt distal end tip 34t toward delivery opening 122, where most preferably treatment element does not traverse opening 122 rather is stopped at pre-implantation site 24p of first assembly shaft 24. Optionally, delivery may be provided by advancing treatment element 10 past opening 122 into the delivery canal. Optionally and preferably first assembly distal end shaft 24 (FIGS. 11D, E) is retracted backward, as shown by directional arrow 15, therein leaving treatment element 10 stationary with respect to the body and implantation site, as shown in FIG. 11E, facilitating a retractive implant delivery manner. Optionally and preferably proximal end 22 remains fixed. Optionally the retraction is provided with a spring (not shown) positioned within first assembly member 20, while implant 10 remains in a fixed position compared to the body until it is deployed and therein placed within the delivery canal (not shown) of target site 308. Optionally, first assembly member 20 may be retracted proximally therein placing implant 10 within delivery canal of target site 308 (not shown). Optionally delivery as depicted with the non limiting optional embodiment of FIGS. 11A-E may be performed with the aid of an external imaging device 320 for example including but not limited to CT, IR, MRI, PET, ultrasound or the like (FIG. 1C). Optionally delivery system 52 may comprise markers (not shown) including but not limited to color markers, metallic markers, radio-opaque markers and the like.

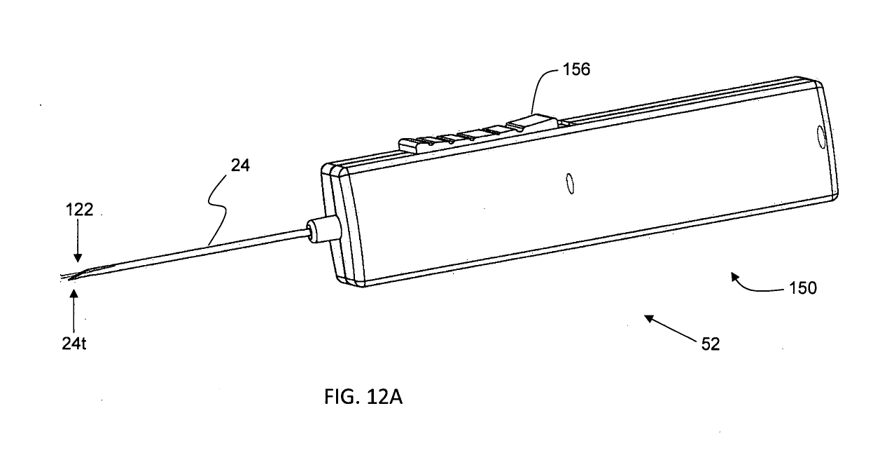

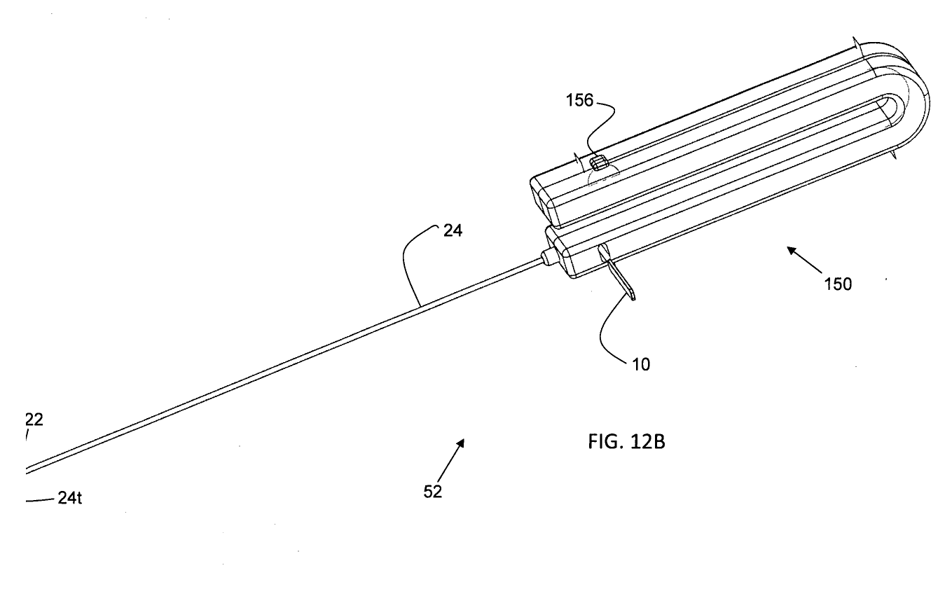

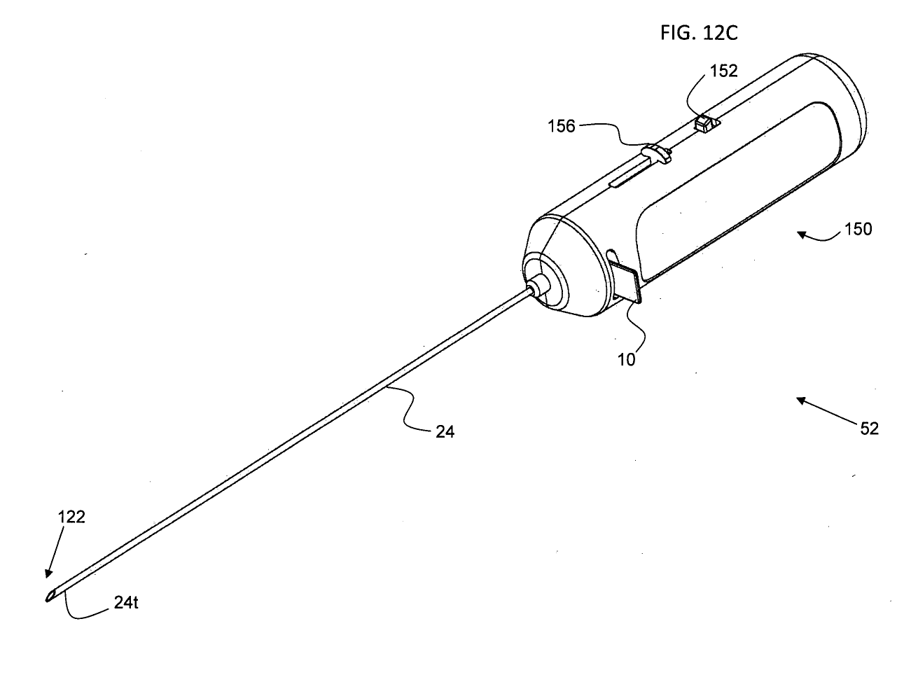

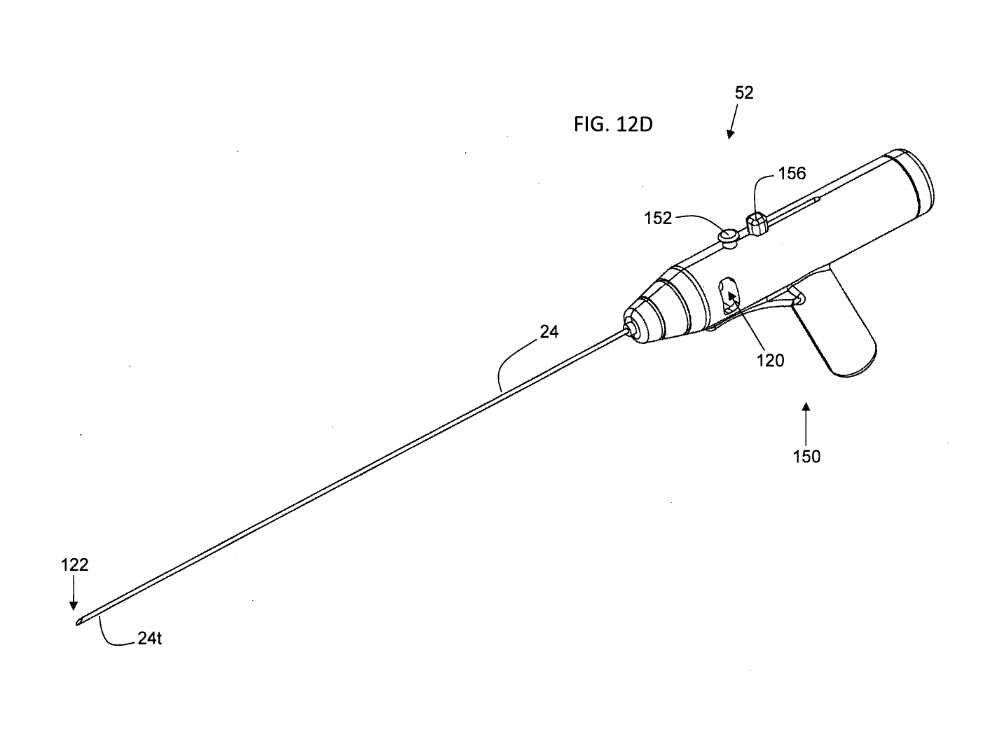

FIGS. 12A-E provide schematic illustrations of non limiting optional embodiment of the implant delivery system 52 according to the present invention optimized but not limited to direct implantation, optionally to be performed with the aid of auxiliary device including introducer or the like. Optionally system 52 may be provided where at least a portion of system 52 for example distal end 24t is coated by a biocompatible sealant.

FIG. 12A provides a perspective view of an optional non limiting system 52 comprising a sharp optionally in the form of a conical or beveled, distal end 24t forming an opening 122, wherein system 52 is preloaded with at least one or more treatment element 10 (not shown). Optionally the length of first assembly member distal end 24 is similar or shorter than that of proximal portion 150. Delivery of an internal and/or preloaded treatment element 10 (not shown) is facilitated with controller 156 that provides for advancing for example by linear step movement or by wheel step (not shown) at least one or more treatment element 10 distally toward distal end 24t. Optionally system 52 may provide for topical delivery of at least one or more treatment element 10 (not shown). Optionally materials of system 52 are selected for a single-use example system 52 is not capable for multiple sterilization and/or can bare low costs of production.

FIGS. 12B-D provided a perspective view of optional non limiting embodiments of system 52 of the present invention, having varying handles shapes that are optionally cylindrical, square, or the like for ease of use for an implant delivery system similar to that described in FIG. 12A, however further comprising a treatment element inlet loading window 120. Preferably inlet window 120 is adept for accepting a treatment element 10 in a plurality of optional forms for example including but not limited to slab, cylinder, block, substrate, foil, fiber, mesh, ring, pill, film or the like. Optionally and preferably a plurality of treatment elements may be cut from a treatment element substrate 10, slab, film, cylinder, foil or the like during delivery, for example in the form of a cutter 152 and delivered to target site 308 as previously described by advancing treatment element 10 distally with controller 156 for example by linear step movement or by wheel step (not shown).

FIG. 12B provides an example of a U shape handle providing a longer distal assembly for example for penetration deeper tissue or target sites compared to systems described above in FIG. 12A. The distal parts (not shown) of the moveable parts for example needle and shaft are optionally and preferably flexible and the distance extension achieved is about twice the distance of FIG. 12A.

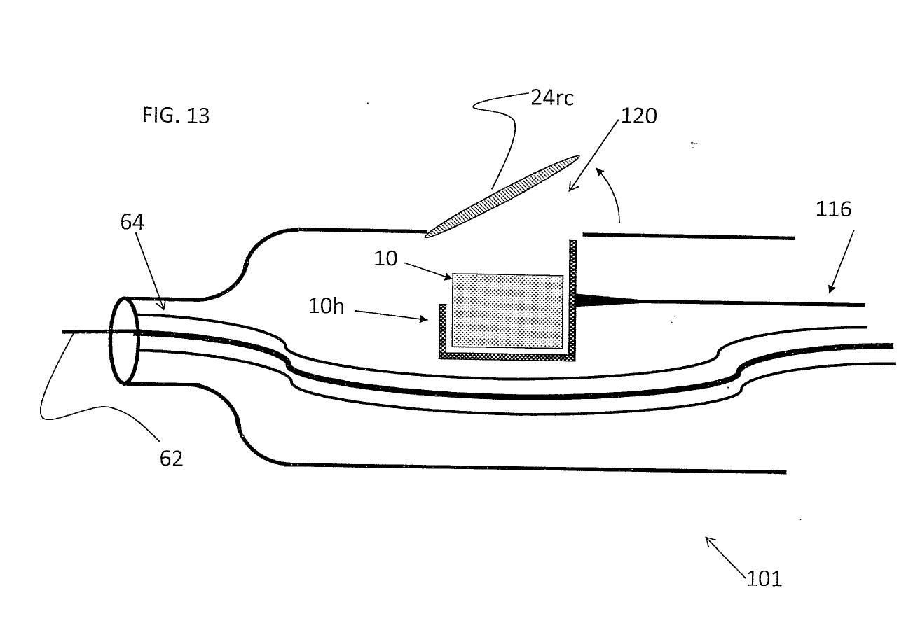

Referring now to FIG. 13 showing a schematic illustration of an optional distal portion 101 of a system 50 according to an optional embodiment of the present invention where implant delivery is facilitated with an auxiliary device in the form of a guide wire 62. Distal portion 101 comprises at least one or more treatment element 10 that may optionally be preloaded into the implant delivery system or optionally may be loaded prior to implant delivery for example through a inlet loading window 120 as previously described in FIG. 4A-J. Optionally guide wire 62 may be disposed in a first or secondary assembly of the system according to the present invention. Optionally and preferably guide wire 62 is associated with distal end portion 101 through a guide wire sheath and/or conduit 64 disposed therein.

Optionally treatment element 10 may be provided in a shape specific to guide wire facilitated implant delivery.

Optionally treatment element 10 may be delivered through any delivery manner previously described, for example side, lateral delivery. Optionally delivery may be facilitated through at least one or more recess disposed on distal end portion 101 for example in the form of a recess disposed on a first assembly housing where implant delivery is provided through a first assembly recess cover 24rc, for example as described in FIG. 8. Optionally any portion of distal end assembly portion 101 may be coated with a biocompatible sealant and most preferably at least recess cover 24rc may be coated with a biocompatible sealant.

Optionally guide wire 62 disposed within conduit 64 may be provided in a substantially linear line. Optionally guide wire 62 may be provided in parts with a curvature preferably provided for increasing the intraluminal space provided within distal end assembly portion 101 optionally to provide for at least one or more treatment element 10. Optionally system 50 may provide for the delivery of at least one or more treatment element 10 in vascular applications, for example including cardiovascular, endovascular, neurovascular, abdomen and peripheral applications or the like. Optionally system 50 may provide for the delivery of treatment element 10 through very narrow arteries and/or veins of diameter typically in the range of about 0.5 to about 2 mm. Optionally system 50 may provide for injecting a fluid in a manner described previously.

Optionally system 50 may provide for the delivery of at least one or more treatment element 10 into the pancreas and/or the liver, with the aid of endoscope including but not limited to duodenal endoscope, where system 50 is inserted through the endoscope working channel optionally of diameter of about 2.8 mm or larger, and then preferably through the major duodenal papilla into the bile duct and/or the pancreatic duct for example over guide wire 62 that was inserted into the duct beforehand, where treatment element 10 is delivered, optionally by side delivery through the walls of these ducts into a tumor localized in the vicinity of these ducts.

Referring now to FIG. 14, shows a flowchart of an optional method for implant delivery of at least one or more treatment elements according to the present invention, where optional implant delivery system 50, 52 is utilized with an optional auxiliary device for example including but not limited to trocar, guiding catheter, catheter, endoscope, endoscope with working channels, endoscope with ultrasound probe, introducer, stepper, port, syringe or the like. For example, the method is as described herein below may be better understood with the illustrative schematic diagram of FIGS. 1A-C.

First in stage 1 the target site is visualized and/or imaged to identify and/or route and/or path to the target site, optionally with an imaging device 320 for example including but not limited to CT, MRI, Ultrasound, IR, PET or the like. Next in stage 2 an access route to target site is planned, for example access route 306, preferably to optimize the access route 306 to target site 308. Next in stage 3, access toward the target site is gained optionally in an indirect manner, stage 3-a for example as depicted in FIG. 1A-B with system 50 utilizing an auxiliary system in the form of an endoscope. Optionally, access may be gained directly, stage 3-b, optionally with a syringe like system 52 as depicted in FIG. 1C, and 11A-E.

For example, in stage 3a an auxiliary device for example an endoscope catheter is associated with a patient 300 and advanced as close as possible to the target site 308. Optionally the type of auxiliary device used to facilitate imagine and delivery of at least one or more treatment element is depended on the target site 308. For example, referring now to FIG. 1A-B target site 308 is found within pancreas 304, where for example an endoscope 310 comprising an ultrasound probe 320 and a working channel is used to provide for visualization of the target site 308. Endoscope 310 is advanced as close as possible to target site 308 through the GI tract resting within the stomach 302 just above target 308. Most preferably ultrasound probe 320 provides an image of the target site 308. Next in stage 4, at least one or more treatment element is associated with the optional system used, 50 or 52, for example a preloaded treatment element in stage 4b or a through an optional inlet loading window in stage 4a. Optionally in stage 4-a a treatment element 10 may be loaded with the implant delivery system depicted in FIG. 4 where a treatment element is loaded through an inlet window 120 as previously described and then associated with working channel opening 316.

Next in stage 5 the implant delivery system according to the present invention is coupled with the auxiliary device, for example endoscope 310 through a working channel opening 316. Next in stage 6 the target site route is implemented where a beveled end tip is driven through the stomach lining to initiate rout 306 toward pancreas 304 and target site 308 where at least one or more treatment elements may be delivered, as shown in FIG. 1B. Optionally as shown in FIG. 1A route 306 may require accessing an intermediate tissue for example the stomach linings may be pierced in order to gain access to target site 308.

Next in an optional stage 7a implant delivery system 50 may be stabilized with at least on or more stabilizing device for example including but not limited to medical balloon, net, filter, mesh, metallic mesh, ring, anchors or the like.

Optionally when the target site is a radial target site disposed with a circumferential delivery site, for example within the esophagus lumen, the target site may be approached intraluminally with an optional system according to the present invention, for example that described in FIGS. 9-10. Once the implant delivery system is stabilized as described in stage 7a, optionally in stage 7b a distal portion of implant delivery system is controllably bent to provide for reaching a radial target site. Optionally an angle may be controllably implemented for example between about 0 degrees to about 120 degrees to provide for reaching the target site.

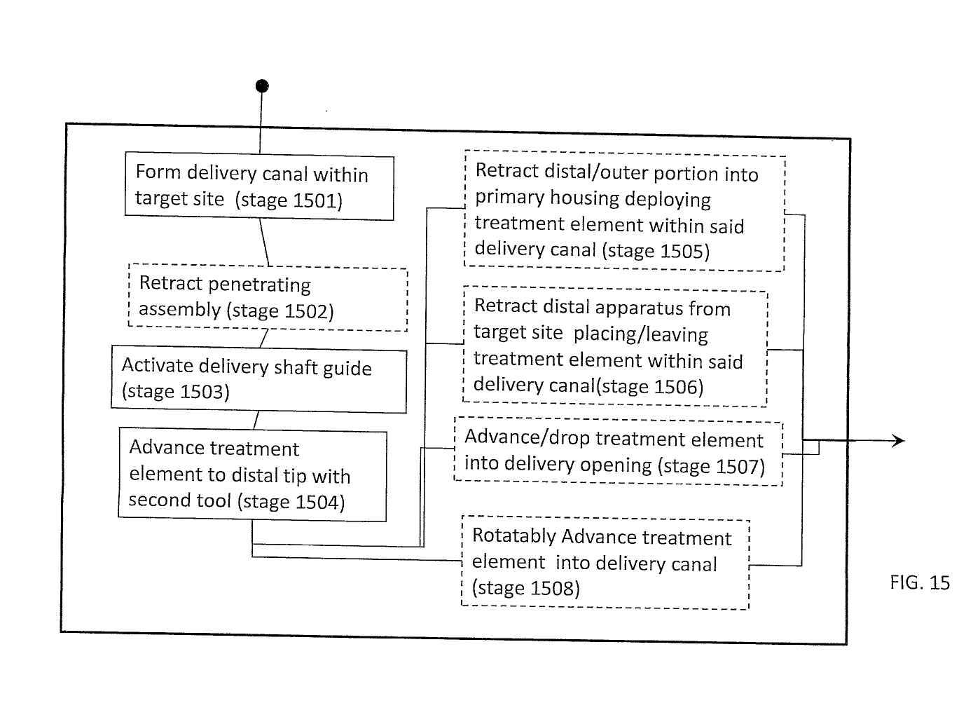

Next in stage 8 treatment element delivery is implemented to delivery at least one or more treatment element at a target site 308, with the distal portion of the implant delivery system according to the present invention. Greater detail of stage 8 is provided in FIG. 15 described below.

Following implantation in stage 9 the implant delivery system is removed proximally from the delivery site while tissue along the delivery route 306 may optionally be sampled, repaired, disinfected, cleaned, closed, sutured or the like if needed to expedite healing. Optionally if a stabilization device was utilized it is preferably deactivate in stage 9, following implant delivery. Next in stage 10 the implant delivery system 50 according to the present invention is removed or disassociated from the auxiliary device, for example by removing system 50 from the catheter, endoscope, endoscope working channel, trocar or the like. Finally in stage 11 the auxiliary device is vacated from the subject for example patient 300 of FIG. 1A.

Referring now to FIG. 15 showing a flowchart of an optional method for the delivery of at least one or more treatment element 10 according to the present invention, implemented with the method described in FIG. 14 showing how the implantation process itself may optionally and preferably is performed. FIG. 15 describes in greater details stages 8 of Figure. First in stage 1051 access is gained toward the delivery site preferably by penetrating the tissue for example a solid tumor with an assembly having a beveled or like sharp edge for example a needle to provide for penetration. Next optionally the penetrating assembly and/or needle 112 is removed in stage 1052. Optionally and preferably removal of the penetrating assembly and/or needle 112 is dependent on the type optional system according to the present invention that is utilized. For example optional embodiments of system 50 according to the present invention for example FIGS. 4A, 11A are optionally provided with non-beveled blunt distal tip and therefore require a penetrating assembly to be inserted and or removed to allow for delivery. For example optional embodiments provided with an integrated beveled, tapered or the like sharp distal end, for example as described in FIGS. 6, 7, 12, do note require retraction or removal of penetrating assembly during stage 1052.

Next in stage 1053 a delivery shaft-guide for example guide 116 as previously described, is activated to facilitated delivery. Optionally shaft-guide activation may for example comprise utilizing an adaptor 130 as described in FIG. 3D; or placement of the shaft-guide within the appropriate assembly as described in FIG. 11C-D. Next in stage 1054 delivery is facilitated with a delivery shaft-guide to urge at least one or more treatment elements distally toward the distal end of system 50 near the delivery site.

Next the delivery manner is provided by selecting at least one of optional stages 1055 to 1058. Preferably the delivery manner is depicted in optional stages 1055-1058 is chosen based on the optional embodiment of the system utilized, or the target site, and the direct or indirect delivery process utilized. Optionally in stage 1055 the distal portion of system is retracted and moved proximally into system 50 to deploy at least one or more treatment element 10 within target site, as previously described in FIG. 5 and FIG. 4A where the distal end 34 or 24 may be retracted.

Optionally in stage 1056 the full system may be retracted proximally to gently drop and/or place at least one or more treatment element 10 into the target site 308, for example as described in FIG. 3.

Optionally in stage 1057 at least one or more treatment element 10 may optionally be dropped, advanced and/or guided and/or gently urged distally crossing distal end of system 50 and into delivery opening 122 out of the distal end of system 50 according to optional embodiment of the present invention with guide-shaft 116. Optionally guide shaft 116 may be controlled manually, mechanically, air pressure or the like device or implementation.

Optionally in stage 1058 at least one or more treatment element 10 may be delivered into the target site 308 with a rotational manipulation of at least one or more assembly comprising implant delivery system 50. Optionally a first optional assembly may be rotated about a second optional assembly to advance at least one or more treatment element 10 into target site 308. Optionally a second assembly member may be rotated about a first optional assembly to advance at least one or more treatment element 10 into target site 308. Following delivery according to at least one manner as described in stages 1055-1058, delivery if followed up as previously described in FIG. 14 from stages 9 an on.

### EXAMPLES

### Example 1

### Locations of Target Tissues, Disease Types, and Implantation Methods

Table 1 presents examples of locations of target tissues, disease types, and the methods used to implant the non-fluid treatment element according to the present invention termed a “LODER” using the systems and method described hereinabove.

TABLE 1

| Location/Target | | |
|---|---|---|
| tissue | Disease | Method |
| Pancreas-head | Pancreatic cancer | Direct-percutaneous |
| | | Duodenal Endoscope |
| | | EUS-NOTES through the |
| | | stomach |
| Pancreas-body | Pancreatic cancer | Direct |
| and tail | | EUS-NOTES through the |
| | | stomach |
| Liver | Hepatoma | Direct-similar to percutaneous |
| | | liver biopsy |
| | | Transvenous-similar to |
| | | Transvenous Liver Biopsy- |
| | | system is inserted via a sheath |
| | | through the jugular vein the |
| | | hepatic veins |
| | | Laparoscopic |
| | | EUS-NOTES through the |
| | | stomach |
| Prostate gland | Prostate cancer | Direct |
| | | Brachytherapy-procedure |
| | | resembles the Permanent Seed |
| | | Implantation, the implantation |
| | | system is inserted through a flexible |
| | | catheter. |
| Bones | Osteomeialitis | Direct, with drill |
| Esophagus | Chrons; Barrett | Endoscopic, with fixation/balloon |
| | Cancer | and bending |
| Cervix | Cervical cancer | Direct, with fixation/anchoring and |
| | | bending |
| Salivary glands | Cancer and | Direct, via canal |
| | inflammation | |
| Heart | Cardiomyoplasty | Via Guiding catheter |
| Lungs | Cancer and | Bronchoscope/Thoracoscope |
| | Tuberculosis | |
| Breast | Cancer | Direct |
| Thyroid | Cancer | Direct |
| Brain | Glioblastoma | Streotactic |
| | | Neurovascular micro-catheter |
| Skin | Melanoma | Direct |
| Peritoneum | Cancer | Laparoscopy/NOTES/Endoscope- |
| | | Implant Anchoring |

### Example 2

### Pancreatic Cancer (Hilar Cholangiocarcinoma)

Pancreatic cancer is an aggressive tumor which is usually diagnosed at late stage. The current estimated of new cases and deaths from pancreatic cancer in the United States in 2008 is 37,680 for new cases and 34,290 for deaths. Carcinoma of the pancreas has had a markedly increased incidence during the past several decades and ranks as the fourth leading cause of cancer death in the United States. Despite the high mortality rate associated with pancreatic cancer, its etiology is poorly understood. Cancer of the exocrine pancreas is rarely curable and has an overall survival (OS) rate of less than 4%. The highest cure rate occurs if the tumor is truly localized to the pancreas; however, this stage of the disease accounts for fewer than 20% of cases. For those patients with localized disease and small cancers (<2 cm) with no lymph node metastases and no extension beyond the capsule of the pancreas, complete surgical resection can yield actuarial 5-year survival rates of 18% to 24%. Improvements in imaging technology, including spiral CT scans, MRI scans, PET scans, EUS examination, and laparoscopic staging aids in the diagnosis and the identification of patients with disease that is not amenable to resection. Complete surgical resection is the only potentially curative option for pancreatic cancer. However, most patients have advanced/metastatic disease at the time of diagnosis, or will relapse after surgery. Systemic chemotherapy is only palliative. Gemcitabine-based therapy is an acceptable standard for unresectable locally advanced/metastatic pancreatic cancer, but average median survival is only 6 months. Pancreatic cancer is the second most frequent gastrointestinal malignancy and carries a dismal prognosis. The current standard of care includes resection, if possible, as well as systemic chemoradiation therapy. Endoscopic ultrasound (EUS) is an established technique for the diagnosis and staging of pancreatic adenocarcinoma. When compared with CT, EUS has been shown to be more sensitive in detecting pancreatic masses (98% versus 56%) and more accurate in tumor staging (67% versus 41%). Interventional EUS via fine needle injection (FNI) for the treatment of pancreatic cancer is a rapidly expanding field. Furthermore, there is a growing interest in neo-adjuvant (anti-tumor therapy prior to surgical resection) approaches to cancer therapy and in particular to pancreatic carcinoma. However, the helpfulness of this approach is questionable due to the low effectiveness of current modalities, which encounter low specificity with systemic side effects. Combining locally administered neo-adjuvant with surgical resection becomes a viable option and could increase the percentage of patients possibly undergoing a surgical resection. Hilar cholangiocarcinoma (or Klatskin tumor) is a rare condition, accounting for less than 1% of all cancers and has no treatment Because of their location these tumors present late and therefore are usually not resectable at the time of presentation. Complete resection of the tumor offers hope of long term survival and of late there has been renewed interest in liver transplantation from deceased donors along with adjuvant therapy. Prognosis remains poor today.

An optional embodiment of the present invention provides for an effective treatment of Pancreatic Cancer and/or Hilar cholangiocarcinoma (Klatskin tumor) without chemotherapy, provided by the method described in FIG. 14 with the an optional system described in FIGS. 2-10. FIG. 1A-B provides a depiction of the system and method adapted for the treatment of pancreatic cancer. Optionally the system of the present invention may be utilized to deliver at least one or more LODER specific for pancreatic cancer adjacent to or more preferably within the pancreatic tumor site 308. Optionally and preferably pancreatic delivery may be facilitated with endoscospe 310 for example a EUS (endoscope ultra-sound). This approach for treating pancreatic cancer provides a non invasive manner for treating pancreatic cancer while avoiding other side effects such as inflammation. The proximal delivery of a non-fluid treatment element 10, also referred to as a LODER, offered by the system and method of the present invention allows for specific and localized targeting of the tumor. The system and method according to the present invention provide for approximating the LODER treatment element 10 to the tumor's 308 microenvironment, while locally delivering a treatment payload targeted the specific type of cancer.

As previously described in FIGS. 1A-B and 14 an endoscope 310 is advanced through the upper GI tract to the stomach 302 just above a pancreatic solid target 308 within pancreas 304. Preferably ultrasound probe 320 is provided to visualize the target area and plan entry route 306 to the targeted pancreatic tumor 308. Once route 306 is devised optionally and preferably endoscope 310 is loaded with an optional system according to the present invention comprising at least one or more treatment element non-fluid treatment element 10, loaded with agent specific for the treatment of pancreatic tumor 308. As can be seen in FIG. 1B route 306 may be provided by piercing and traversing stomach wall 302 to gain access to pancreas 304, with an optional implant delivery system according to the present invention, most preferably with a needle 112 or the like penetrating assembly having a sharp distal end according to an optional embodiment of the system according to the present invention to penetrate stomach wall 302. Preferably pancreas 304 is then penetrated optionally and preferably with the same penetrating assembly or optionally with a second penetrating assembly. Optionally and preferably pancreatic tumor 308 is penetrated with a penetrating assembly comprising a beveled distal end of about 18 gauge to about 21 gauge. Once tumor 308 is penetrated wherein most preferably at least one or more treatment element 10 most preferably provided in the form of pancreatic tumor specific LODER is delivered optionally by placing or urging the LODER within and/or adjacent to the tumor 308 optionally with a guiding shaft 116, as previously described in FIG. 15.

Optionally pancreatic delivery and specifically delivery to the pancreatic head may be facilitated with another type of endoscospe for example a duodenal endoscope, used for example in ERCP procedures, having a side-looking view and an elevator that capable to bend instruments that are advanced within the working channel, including the delivery system described in this invention, up to about 90°, and thereby enables the entrance at the Major duodenal papilla to the bile duct and the pancreatic duct. Optionally pancreas 304 is then penetrated with a system described by FIG. 13. Another approach that may be used is a direct approach where a system optionally as described in FIGS. 11A-D and 12A-D is used. Such an approach resembles a direct biopsy procedure that is performed with the aid of CT imaging. Usually an introducer (sometimes called “coaxial” is inserted first, and then the biopsy needle. Such an introducer is used also here in this direct approach of implant delivery. The first assembly member distal portion 24 is inserted to the body at the access point and through a penetration passage previously selected based on CT imaging.

### Example 3

### Esophageal Cancer

One specific example of esophageal cancer is Barrett's esophagus referring to an abnormal change, metaplasia, in the cells of the lower end of the esophagus, thought to be caused by damage from chronic acid exposure, or reflux esophagitis. Barrett's esophagus is found in about 10% of patients who seek medical care for heartburn, gastroesophageal reflux. It is considered to be a premalignant condition and is associated with an increased risk of esophageal cancer.

An optional embodiment according to the system and method of the present invention provides for the treatment of such esophageal cancer. Optionally indirect delivery of at least one or more treatment elements 10 with the aid of an auxiliary device such as an endoscope or catheter according to optional embodiment of the present invention as previously described may be provided for a comprehensive treatment. Optionally the system for radial delivery of at least one or more treatment element specific for esophageal cancer comprising siRNA for example targeted to K-ras, cyclin D1 or c-erb-2. Optionally and preferably a plurality of treatment elements 10 will be required to treat a plurality of treatment sites disposed radially about the esophagus. Radial delivery as previously described in FIG. 14 may be utilized to delivery a plurality of treatment element comprising siRNA optionally targeted to K-ras, cyclin D1 or c-erb-2. Optionally the system is bent for example as shown in FIG. 9A-B. Optionally the angle of bending is varied from about 30 degree to 90 degree relative to the longitudinal axis of the system. Optionally the system may be stabilized for example using a stabilization device as described in FIG. 10A-C, within a luminal circumferential delivery site as seen in an esophagus. As previously described the system would be stabilized with an optional stabilizing device for example medical balloon, semi-compliance balloon, net, filter, mesh, metallic mesh, ring, anchors or the like to stabilized the implant delivery system against the esophageal walls in the vicinity of the radial target area. For example the stabilization would optionally be performed to the esophagus walls at the location of about 10-20 mm above the targeted treatment area. Optionally stabilization may be provided with a temporary anchoring device where the delivery system according to the present invention is temporarily anchored to the esophageal wall. Radial delivery is provided as previously described in FIG. 14.

While the invention has been described with respect to a limited number of embodiments, it will be appreciated that many variations, modifications and other applications of the invention may be made.

1-4. (canceled)

5. The system of claim 22 wherein said assembly distal end is provided in a shape chosen from the group consisting of blunt, tapered, beveled, oval, spherical, blunt with curved edges, conical, pyramidal and pyramid like having a plurality of faces, any combination thereof.

6-13. (canceled)

14. The system of claim 22 wherein said at least one or more opening provides for an implant delivery manner chosen from the group consisting of lateral delivery, side delivery, angular delivery, end delivery, retractive delivery, rotational delivery and pull back delivery, radial delivery, injection delivery, push, placing, deploying, dropping, aspiration, air pressure, vacuum, spring pressure, hydraulic, pressure differential, mechanical, manual manipulation, rotational, screw threading, helical thrust affix, stick, glue, pull, pull out, blow, guide wire assisted, spring assisted, magnetic force assisted, through an opening, through a covering and any combination thereof.

15-18. (canceled)

19. The system of claim 22 wherein said inlet loading window is configured to provide for receiving said at least one or more treatment element within said at least one or more inlet loading window utilizing method chosen from the group consisting of vacuum pulling, magnetic pulling, holding, stowing, maintaining, retaining and any combination thereof, until deployment of said treatment element; and wherein said configuration is chosen from the group consisting of door, separator, cover, auxiliary device, and any combination thereof.

20-21. (canceled)

22. A system for the delivery of at least one or more treatment element to a target site wherein at least one of said treatment element is provided in the form of a non-fluid treatment element comprising a nucleotide based agent, said system comprising an assembly having a distal end and a proximal end; and at least one or more opening for delivering said at least one treatment element to said target site,
wherein said assembly further comprises at least one or more treatment element inlet loading window and wherein said window is provided to receive a treatment element wherein said treatment element is not shape specific therein provided in raw form chosen from the group consisting of slab, block, cylinder, substrate, foil, fiber, mesh, ring, film, any combination thereof.

23. (canceled)

24. The system of claim 22 wherein said treatment element is provided having a predetermined and consistent geometric parameters chosen from the group consisiting of size, shape, radius, height, width, angle, thickness, volume, surface area, circumference, ellipticity, oval, polygon, curvature, hole dimension, void, waviness, roundness, layer spacing, and mesh spacing.

25-32. (canceled)

33. The system of claim 22 wherein said assembly further comprises at least one or more partition, separator or septum.

34. The system of claim 22 further comprises at least one or more auxiliary devices chosen from the group consisting of a needle, guide, aspiration needle, hypodermic needle, biopsy needle, thermal needle, cryo-needle, balloon, guide wire, stapler, scalpel, anchoring part, drill, heater, stereotactic tools, camera, imaging device electrode, ultrasonic probe, IR transceiver transmitter/transceiver, wireless transmitter/transceiver, flushing device, regional anesthesia device, cleaner, suction device, graspers scissors, hook, ablation device, screw, pad, sticker-pad, supporting ring, embolic filters, plunger, adaptor, needle adaptor, balloon, septum/partition, net, filter, mesh, metallic mesh, ring, spring, anchors, stabilization device.

35-37. (canceled)

38. The system of claim 22 wherein said assembly further comprises a flexible or bendable portion providing for introducing an angular bending and or movement from about 0 degrees to about 120 degrees.

39. The system of claim 22 wherein said system is adapted to work with at least one auxiliary device chosen from the group consisting of: trocar, guiding catheter, catheter, endoscope, endoscope with working channel, endoscope with ultrasound probe, borescope, introducer, stepper, sheath, port and syringe.

40-43. (canceled)

44. The system of claim 22 wherein said distal end may be coupled to said proximal end with corresponding coupling members chosen from the group consisting of recess and latch, connectors including male connector and female connector, threading, wire, hook and loop and corresponding threading, connecting tube, chain, braid, snaps, magnetic and glue.

45. The system of claim 22 wherein said distal end comprises a lid.

46. The system of claim 22 wherein any portion of said system and may be substantially hermetically sealed with a biocompatible sealant.

47. The system of claim 22 wherein said distal end is provided as a conical or sharp shaped grasper claw that may be controllably manipulated to form an open or closed configuration comprising at least two conical or sharp shaped members and wherein each of said member correspond and engage one another to form said conical or sharp shaped grasper claw.

48. The system of claim 22 further comprising a conduit for delivering flowing materials chosen from the group consisting of fluid, gel, sol-gel, foam, suspension, hydrogel, micro-particles, nano-particles, powder and solution.

49. (canceled)

50. The system of claim 22 wherein said assembly further comprises markers, wherein said marker is chosen from the group of visible markers, visual markers, and radio-opaque compounds, chosen from the group consisting of heavy metals, gold, platinum, titanium, polymer enrichments, Barium Sulfate (BaSO4), ultrasound markers, MRI markers, fluorine-19, IR markers, metallic markers active markers, fluid enrichment markers and air enrichment markers.

51-52. (canceled)

53. The system of claim 22 wherein at least one segment of at least one part of said assembly is coated by coating selected from a group including friction reducing, hydrophilic, cell growth enhancing, anti-microbial, anti-thrombogenic, anti-cell adhesion, anti-cell proliferation, radio-opaque, non-immunogenic, non-allergic, any combination thereof.

54. The system of claim 22 wherein said assembly is guided over a guidewire.

55. The system of claim 22 wherein said treatment element is targeted to a target site chosen from the group consisting of pancreas; breast; prostate; liver; gallbladder; spleen; kidney; lymph nodes; salivary glands; peridontal tissue; intra-vaginal; endocrine gland; brain; joint; bone; oral cavity; gastrointestinal system (GI tract); biliary system; respiratory systems, cardiovascular system, artery; vein; heart, any part of the vascular system; uterus, uterine cervix; fallopian tubes, ovaries, female reproductive tract, penis, gonads, male reproductive tract; ureter or urethra; the basal ganglia, white and gray matter; the spine; active and chronic inflammatory joints; the dermis; sympathetic and sensoric nervous sites; intra osseous; acute and chronic infection sites; ear; Intra-cardiac; cardiovascular system, epicardiac; urinary bladder; parenchymal tissues; Intra-ocular; Brain tissue; Brain ventricles, intracranial space, a cavity, mouth, pharynx, esophagus, stomach, small intestine or a portion thereof, appendix, large intestine (colon) or a portion thereof, rectum or anus, auditory system, labyrinth of the inner ear, vestibular system, nose, nasal conchae (also called turbinates), pharynx, larynx, trachea, bronchi, lungs, auditory tube, and the muscles of inspiration (the diaphragm and external intercostal muscles), skull, spinal canal, thoracic cavity, abdominal cavity, and pelvic cavity.

56. A method for the delivery of at least one or more treatment element to a target site with the system of claim 22, the method comprising: a. planning access route approach to said target site; b. receiving at least one or more treatment element, provided in raw form, into the inlet loading window; c. advancing said system toward the target site and gain access to said target site through said access route with said system; d. forming a delivery canal within said target site; e. delivering said at least one or more treatment element into said delivery canal; and f. vacate-vacating said delivery canal.

57. (canceled)

58. The method of claim 56 wherein during stage (c) said system is associated with at least one auxiliary device chosen from the group consisting of an endoscope, trocar, guiding catheter, catheter, endoscope, endoscope with working channel, endoscope comprising an ultrasound probe, sleeve, stepper and introducer, wherein said auxiliary device is advanced toward target site to further identify said target site; and wherein said system is associated with said at least one auxiliary device, for facilitating said treatment element delivery

59-65. (canceled)
